SEQUENCE LISTING (10 sequences)

SEQ ID NO: 1 - protein, 5 aa, Artificial
  Synthetic peptide
YIGSR

SEQ ID NO: 2 - protein, 4 aa, Artificial
  Synthetic peptide
REDV

SEQ ID NO: 3 - protein, 5 aa, Artificial
  Synthetic peptide
GRGDY

SEQ ID NO: 4 - protein, 6 aa, Artificial
  Synthetic peptide
GREDVY

SEQ ID NO: 5 - protein, 4 aa, Artificial
  Synthetic peptide
RGDS

SEQ ID NO: 6 - protein, 5 aa, Artificial
  Synthetic peptide
LRGDN

SEQ ID NO: 7 - protein, 5 aa, Artificial
  Synthetic peptide
PDSGR

SEQ ID NO: 8 - protein, 4 aa, Artificial
  Synthetic peptide
RGDT

SEQ ID NO: 9 - protein, 4 aa, Artificial
  Synthetic peptide
DGEA

SEQ ID NO: 10 - protein, 5 aa, Artificial
  Synthetic peptide
IKVAV

(19) United States
(12) Patent Application Publication
(10) Pub. No.: US 2008/0299601 A1
(43) Pub. Date: Dec. 4, 2008

(54) Adhering Surfaces

(75) Inventors: Richard Fike, Clarence, NY (US); Robert Burrier, Clarence Center, NY (US)

Correspondence Address: INVITROGEN CORPORATION;C/O INTELLEVATE, P.O. BOX 52050, MINNEAPOLIS, MN, 55402, US

(73) Assignee: INVITROGEN CORPORATION, Carlsbad, CA (US)

(21) Appl. No.: 12/047,997

(22) Filed: Mar. 13, 2008

Related U.S. Application Data
(60) Provisional application No. 60/895,009, filed on Mar. 15, 2007.

(51) Int. Cl. C12Q 1/02; C12N 5/06

(52) U.S. Cl. 435/29; 435/402

(57) ABSTRACT

The present invention relates, in part, to compositions useful for cell culture having one surface in adherence to another surface, e.g., a cell culture matrix in adherence to a surface. The present invention also relates, in part, to methods of adhering one surface to another surface, e.g., adhering a cell culture matrix to a surface, and compositions relating to such methods. The present invention also provides in part, methods for adhering a cell to a surface. Related methods are also provided for determining the effect of at least one compound on a cell(s).

### 1. CROSS REFERENCE TO RELATED APPLICATIONS

This application claims the benefit of U.S. Provisional Application No. 60/895,009, filed Mar. 15, 2007, the entire disclosure of which is incorporated herein by reference.

### 2. FIELD OF THE INVENTION

The present invention provides, in part, to methods for adhering one surface to another surface. The present invention also relates, in part, to methods of producing 3-D cell culture matrices; methods of growing cells and methods of determining effects of at least one compound on at least one cell. The invention also relates, in part, to methods of adhering two negatively charged or two hydrophobic surfaces. The invention provides methods for adhering a cell to a surface, e.g., under culturing conditions.

### 3. BACKGROUND OF THE INVENTION

Three dimensional matrices or scaffolds for cell culture are useful for culturing cells and/or for certain applications. In some cases, cells grown in three dimensional properties exhibit different characteristics than when grown in two dimensional culture and/or without a matrix. These 3 dimensional cell culture methods can be used to investigate the behavior of cells in a 3-dimensional framework in vitro (Jain and Tandon Biomaterials 11:465-472 (1990); Doane and Birk Exp Cell Res. 195(2):432-42 (1991)). In some applications, these matrices are designed to serve as analogues of an extracellular matrix in order to provide a suitable substrate for cell attachment to enable certain anchor-dependent processes such as migration, mitosis, and matrix synthesis (Folkman and Moscona Nature 273:345-349 (1978)). In this regard, it is considered that such analogues of the extracellular matrix may be able to modulate cell behavior in a similar fashion to the way in which the native extracellular matrix does (e.g., see Madri and Basson, Lab. Invest. 66:519-521 (1992)), it being believed that the chemistry of these analogues, as well as their pore characteristics such as percentage porosity, pore size and orientation, may influence the density and distribution of the cells within the matrix and thereby affect the regeneration process when these analogues are used in transplantations. In some applications, the cells can be grown as spheroids.

Bioresorbable matrices (e.g., sponges) can also provide a temporary scaffolding, e.g., for transplanted cells, and thereby allow the cells to secrete an extracellular matrix of their own. The macromolecular structure of these sponges can be selected so that they are completely degradable and are eliminated, once they have achieved their function of providing the initial artificial support for the newly transplanted cells. Typically, these sponges for use in cell transplantations may be highly porous with large surface/volume ratios to accommodate a large number of cells. They will also usually be biocompatible, e.g., non-toxic to the cells they carry and to the host tissue into which they are transplanted. Examples of matrices for growing cells are known in the art. For example, matrices comprising polysaccharides are described in U.S. Pat. No. 6,425,918.

Some porous matrices for cell culture are based on natural polymers such as collagen, or synthetic polymers from the lactic/glycolic acid family. Other synthetic biodegradable foams based on poly(D, L-Lactic-co-glycolic acid) have been developed as scaffolds.

One problem with some matrices is that they do not adhere well to typical and/or commercial tissue culture vessels, e.g., 24- or 96-well plates. Some embodiments of the present invention provide methods and compositions related to adhering and/or enhancing adherence of a matrix for cell culture to a tissue culture vessel.

Citation or discussion of a reference herein shall not be construed as an admission that such is prior art to the present invention.

### 4. SUMMARY OF THE INVENTION

The invention relates, in part, to methods and compositions for attaching at least one material to another. In particular embodiments the invention relates, in part, to methods of adhering materials (e.g., matrices or cells) to surfaces, e.g., wherein the material and surface exhibit like charges. Some related methods comprise coating a first or second surface with a charged molecule and contacting the first surface with the second surface. Some embodiments of the invention provide methods comprising incorporating into a first or second surface a charged molecule and contacting the first surface with the second surface. In some embodiments, the first and second surface have a similar charge (e.g., negative) and the charged molecule has a charge opposite (e.g., positive) of the first and/or second surface. Therefore, the invention also provides compositions comprising a first surface, a second surface and/or a charged molecule. The invention also provides compositions for preparing such surfaces, as well as, compositions which contain these surfaces.

The invention also provides methods and compositions for indirectly attaching a cell to a surface through an intervening cell culture matrix. The invention also relates, in part, to methods for producing a cell culture matrix. Some embodiments comprise coating one or more first surfaces with a charged (e.g., positive or negative) molecule and contacting the one or more first surfaces with one or more second surfaces, e.g., wherein the cell culture matrix comprises the second surface. Some embodiments comprise a first surface coated with a charged molecule and a second surface adhering to the first surface, wherein a cell culture matrix comprises the second surface. In many instances, the second surface will be negatively or positively charged. In some instances, the charge of the second surface will be the opposite charge of the first surface.

Additionally the invention provides, in part, methods for culturing cells on a cell culture matrix. Some methods of the invention comprise: (a) coating a first surface with a charged (e.g., positively) molecule; (b) contacting the first surface with a second surface of the cell culture matrix; and (c) contacting the cells with the cell culture matrix under conditions suitable for culturing the cells.

In some embodiments of the invention, a first surface and/or a second surface is negatively charged. In some embodiments, a first surface and/or a second surface is a hydrophobic or hydrophilic surface. In some embodiments of the invention, a first surface is a portion of a surface of a tissue culture vessel.

Some methods of the invention involve adhering two surfaces in the presence of a liquid. Some methods of the invention involve adhering two surfaces in the absence of a liquid. In some embodiments, a cell culture matrix is employed which is a 3-dimensional cell culture matrix. The invention also provides compositions comprising a cell culture matrix.

In some embodiments of the invention, a positively charged molecule is used which is selected from the group consisting of polyallylamine, polyvinylamine, chitosan, polybutylamine, polyisobutylamine, polyethyleneimine, polyalkyleneamine, polyazetidine, polyvinylguanidine, poly(DADMAC), cationic polyacrylamide, polyamine functionalized polyacrylate, and combinations thereof. In some embodiments, a positively charged molecule is used which is a polyamine. In some embodiments, a positively charged molecule is used which is a chitosan (e.g., chitosan HCl) or a glucosamine-N-acetyl glucosamine polymer.

In some embodiments of the invention, a polyamine used in the practice of some embodiments of the invention has an average molecular weight of between from about 5,000 to about 1,000,000. In some embodiments, a polyamine is used which is a homopolymer, heteropolymer or a copolymer. In some embodiments, a polyamine is used which comprises between from about 2 to about 10,000 nitrogen atoms per molecule.

In some embodiments of the invention, a charged molecule is cross linked prior to, during or after coating. In some embodiments, cross linking a positively charged molecule comprises contacting the positively charged molecule with carbodiimide for example. In some embodiments, a cross linking agent is used to cross link a polyamine.

In some embodiments of the invention, the second surface is a cell culture matrix. In some embodiments, a cell culture matrix comprises at least one of the following: a polyanionic polysaccharide polymer, an alginate, a gellan, a gellan gum, a chitosan (e.g., a xanthan chitosan), polyethylene glycol (PEG), polyvinyl-pyrrolidone (PVP), a calcium phosphate, a polyglycolic acid (PGA), a poly(1-lactic co-glycolic acid (PLGA), PGA/PLGA combinations, a silk, a polypeptide matrix, a collagen, a laminin, a gelatin, a carrageenan, or combinations of collagen, laminin, gelatin. In some embodiments, a cell culture matrix comprises a polysaccharide. In some embodiments, a cell culture matrix comprises alginate. In some embodiments, a cell culture matrix is a sponge. In some embodiments, a sponge comprises alginate.

In some embodiments of the invention, coating of a surface comprises contacting the surface with a positively charged molecule in a solvent. In some embodiments, coating comprises at least two positively charged molecules or at least two polyamines. In some embodiments, a coating solvent is water, an alcohol, or a glycol. In some embodiments, the glycol is methanol, ethanol, ethylene glycol, propylene glycol, or mixtures thereof. In some embodiments, a coating solvent comprising a positively charged molecule is contacted with a surface for a period of time between from about 1 second to about 1 week. In some embodiments, a solvent comprising a positively charged molecule is removed from the surface using aspiration and/or pipetting. In some embodiments, a solvent comprising a positively charged molecule is removed from the first surface using multiple aspiration and/or multiple pipetting. In some embodiments, a solvent comprising a positively charged molecule is removed from the surface and the surface is contacted with a solvent that does not contain the positively charged molecule.

In some embodiments of the invention, cell culture compositions used in the practice of the invention (e.g., matrices) comprise at least one biologically active molecule, e.g., a growth factor, a cell adhesion molecule, an integrin, a cell attachment peptide, a peptide, a growth factor, an enzyme, a proteoglycan or a polysaccharide. Some embodiments of the invention, comprise adding a cell culture matrix solution to a surface and drying the cell culture matrix solution to form a cell culture matrix. In some embodiments, the drying comprises freeze drying. In some embodiments, a cell culture matrix solution comprises alginate. The invention also provides compositions for preparing such cell culture compositions, as well as, the cell culture compositions themselves.

The invention further provides, in part, methods for determining an effect of at least one compound on a cell. Such methods include those which comprise: (a) coating a first surface with a charged (e.g. positive or negative) molecule; (b) contacting the first surface with a second surface of a cell culture matrix; (c) contacting cells with the cell culture matrix under conditions suitable for culturing the cells; (d) contacting the cells with the at least one compound; and (e) determining or detecting the effect or lack of effect of the at least one compound on one or more of the cells. In some embodiments, the at least one compound is a small molecule, an organic molecule, a drug, a protein, a nucleic acid, an antibody, a siRNA, a RNAi, a ligand for a receptor, and a ligand for a G-protein couple receptor. In some embodiments, the cells used in methods described above and elsewhere herein are contacted with at least two compounds. In some embodiments, (e) comprises detecting apoptosis and/or cell death; a metabolic change; a change in cellular cAMP levels; a change in cellular calcium levels; a change in levels of a cellular receptor; and/or a change in levels of a GPCR, e.g., on the cell surface.

The invention provides, in part, methods for producing a cell culture matrix. Such methods include those which comprise contacting a first surface with the cell culture matrix. In some embodiments, a cell culture matrix comprises a charged (e.g., positive) molecule. Some embodiments of the invention provide methods for culturing cells on a cell culture matrix. Such methods include those which comprise: (a) contacting a first surface with the cell culture matrix and (b) contacting the cells with the cell culture matrix under conditions suitable for culturing the cells, wherein the cell culture matrix comprises a positively charged molecule. The invention additionally provides methods for determining an effect of at least one compound. Such methods include those which comprise: (a) contacting a first surface with the cell culture matrix; (b) contacting the cells with the cell culture matrix under conditions suitable for culturing the cells; (c) contacting the cells of (b) with the at least one compound; and (d) determining the effect or lack of effect on the cell, wherein the cell culture matrix comprises a positively charged molecule.

The present invention also relates, in part, to methods of adhering a cell to a surface. Some embodiments comprise coating a surface with a polyallylamine and contacting the coated surface with a cell. In some of these embodiments, a cell is grown in 2-D culture. In some embodiments, a cell has a greater adherence to the coated surface as compared to the uncoated surface. Some embodiments include culturing a cell while the cell is contacted with the coated surface. Some embodiments comprise contacting a cell with the coated surface under conditions suitable for culturing the cell. Some embodiments of the invention provide methods for determining an effect of at least one compound on a cell. Some embodiments comprise: (a) coating a first surface with a positively charged molecule; (b) contacting the first surface with the cell under conditions suitable for culturing the cells; (c) contacting the cells with the at least one compound; and (d) determining or detecting the effect or lack of effect of the at least one compound on the cell.

### 5. BRIEF DESCRIPTION OF THE FIGURES

For the purpose of illustrating the invention, there are depicted in the drawings certain embodiments of the invention. However, the invention is not limited to the precise arrangements and instrumentalities of embodiments depicted in the drawings.

FIG. 1 shows results for an Alamar Blue analysis at day 5. (PAA-good=no noticeable yellowing; PAA*OK=wells with barely discernable yellowing; and PAA*Bad=wells with obvious yellowing of medium post-reconstitution)

FIG. 2 shows an example of a method for producing a cell culture matrix comprising alginate.

FIGS. 3A and 3B shows structures of examples of positively charged molecules that can be utilized in some embodiments of the invention.

FIG. 4 depicts interactions between an alginate matrix, a polyallylamine and a polystyrene sulfonate.

### 6. DETAILED DESCRIPTION

### Definitions

The term “adhere” refers to an attraction between two surfaces. The surfaces can be attracted due to, inter alia, ionic interactions, van der Walls forces/interactions, hydrophobic interactions and/or covalent interactions. The term adhere or adhering also includes, but is not limited to, holding in place, inhibiting movement, inhibiting repositioning, and/or inhibiting detachment. When referring to enhanced or enhancement of adherence means that two surfaces adhere or are attracted better under one condition than another, e.g., in the presence of a molecule as compared to the absence of a molecule. Methods for determining an enhanced or increased adherence are known in the art (e.g., see U.S. patent application Ser. No. 10/805,536) and examples are provided herein.

### Cell Culture Matrices

The terms “cell culture matrix” and “cell culture scaffold” are used interchangeably and refer to a matrix, which cells can grow on and/or in. In some embodiments of the invention, cells will grow within the matrix, e.g., within pores of the matrix. In some embodiments, cells will grow on the matrix. In some embodiments, cells will attach to the matrix. In some embodiments, the cells will grow as spheroids within the cell culture matrix. In some embodiments, a cell culture matrix is 3-dimensional. 3-D cell culture matrices are known in the art, e.g., see U.S. Pat. No. 6,793,675.

Cell culture matrices are known in the art and include, but are not limited to, solid or gel matrices. In some embodiments, the invention utilizes a solid matrix. In some embodiments, the invention utilizes a gel matrix. In some embodiments, a matrix is not a gel matrix. In some embodiments, a matrix is not a solid matrix.

Examples of cell culture matrices include, but are not limited to, those comprising alginate, e.g., alginate sponges. Examples of cell culture matrices are described in, for example U.S. Pat. Nos. 6,793,675, 5,716,404, 6,586,246 and 6,872,387 and PCT Publication Nos. WO 04/082728, WO 00/55300 and WO 06/118554.

In certain embodiments, synthetic matrices (e.g., synthetic polymer matrices) may be used. Examples of such synthetic matrices are polylactic acid (PLA) polymer matrices, polyglycolic acid (PGA) polymer matrices and polylactic acid-polyglycolic acid (PLGA) copolymer matrices including stereoisomeric forms thereof. Chemically, these may also be termed poly-(L-lactic acid), PLA or PLLA, and poly-(D,L-lactic acid), PDLLA. PLGA may also be written poly-(D,L-lactic-co-glycolic acid). In some embodiments, a matrix comprises at least one compound selected from the group consisting of poly(vinyl alcohols), poly(alkylene oxides) particularly poly(ethylene oxides), polypeptides, poly(amino acids), such as poly(lysine), poly(allylamines), poly(acrylates), modified styrene polymers such as poly(4-aminomethylstyrene), polyesters, polyphosphazenes, pluronic polyols, polyoxamers, poly(uronic acids) and copolymers, including graft polymers thereof.

Matrices of the invention may take various forms including, but not limited to fiber matrices, tubular matrices, hydrogel matrices and sponge matrices. In some embodiments, a sponge matrix comprises a polysaccharide, PLA, alginate, silk and/or polyvinyl alcohol (PVA). Further examples of synthetic matrices are those comprising polyanhydrides, polyesters, polyorthoesters, and poly(amino acids), polypeptides, polyethylene oxide, polyphosphazenes, various block copolymers, such as those consisting of ethylene oxide and propylene oxide (e.g., Pluronic surfactant; BASF Corp.), and blends of polymers from this group and blends with other polymers. Ceramics, such as calcium phosphate matrices, may also be employed in the present invention. Cell culture matrices may be homopolymers or heteropolymers.

Any compatible polymer is useful herein, and the selection of the specific polymer and acquisitions or preparations of such polymer are conventionally practiced in the art. See, e.g., The Biomedical Engineering Handbook, ed. Bronzino, Section 4, ed. Park.

Cell culture matrices have a very wide range of uses, for example, they may also be used for, but are not limited to, the in vitro culturing of plant cells and algal cells (e.g., microalgae); for the in vitro support of mammalian oocytes, e.g., for the purposes of in vitro fertilization of these oocytes; for the culture of eukaryotic cells; for the culture of embryonic stem cells; and hence also for the storage of these embryonic stem cells, eukaryotic cells, plant cells, algae, and fertilized oocytes. In some instances, cell culture matrices are used to proliferate and/or culture cells in vitro. In some cases, the unique architecture of a cell culture matrix provides an environment wherein stem cells can be seeded in an undifferentiated state and can be differentiated into a target cell. In some embodiments, the choice of the extracellular matrix coating facilitates differentiation of the cell, e.g., to a target cell. However, essentially any type of cell can be seeded, attached, culture, and/or proliferated in a cell culture matrix, e.g., as described herein. Cell culture matrices provide 3-D cell culture models for use in many research fields, such as toxicology, drug development, cancer and stem cell research, development and morphogenesis, tissue and organ engineering, heart disease, diabetes, and Alzheimer's disease.

Additionally, cell culture matrices may also be used: as drug delivery vehicles (e.g., by way of carrying genetically engineered or natural cells which produce a desired product or drug which is produced in these cells and released to the host from the site at which the sponge was implanted, or the cells are capable of producing and releasing to the surrounding tissue one or more regulatory proteins which direct the production of a desired cellular product in the cells of the tissue surrounding the implant); for the production of therapeutics and/or recombinant proteins; and to deliver various viral vectors, non-viral vectors, polymeric microspheres, liposomes, which encode or contain therapeutic products or drugs of choice that it is desired to administer to the host tissue or organ in which the implant is placed. All of these viral vectors, non-viral vectors, polymeric microspheres and liposomes may be prepared as known in the art to encode or to contain a very wide range of desired agents, for example, various enzymes, hormones and the like, and may be inserted into the cell culture matrix at the time of preparation of the matrix or following the preparation of the matrix. Cell culture matrices are suitable for many cell-based screening and drug discovery procedures, including Multicellular Tumor Spheroid Assays (MCTS), hepatocyte and cardiomyocyte organogenesis studies, co-culture studies, high-throughput (e.g., drug) screening assays, and embryonic stem cell differentiation.

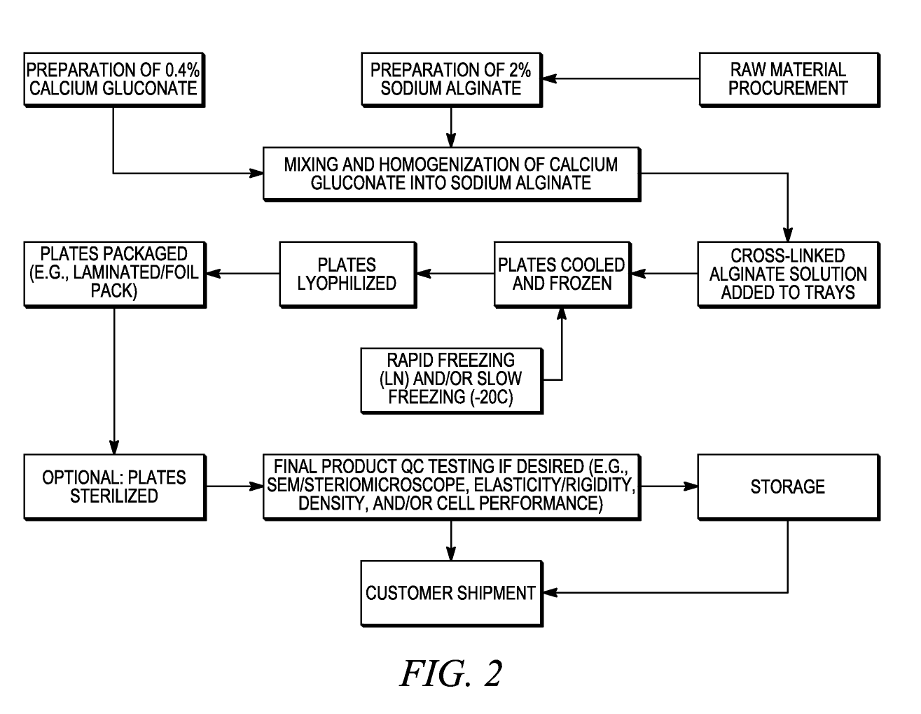

Cell culture matrices of the invention may be any shape suitable for the particular in vitro, ex vivo or in vivo application. For example, a suitable shape can be produced utilizing freeze-drying techniques. In some embodiments, a cross-section may be round, elliptical, star shaped or irregularly polygonal, depending on the application. In some embodiments, a cell culture matrix may be nose shaped, cube shaped, cylindrical shaped and the like, e.g., see FIG. 2 of U.S. Pat. No. 6,425,918. Cell culture matrices of the invention may be used, for example, for nerve, lung, liver, bone, cartilage, and/or soft tissue repair. The scaffold itself may be molded by the selection of a suitable vessel (e.g., a tissue culture vessel) in the methods of preparation or cut or formed into a specific shape that is desired or applicable for its end usage. In some embodiments, a particular shape is achieved by pouring an initial polysaccharide solution into an appropriately shaped vessel having the desired shape and performing the gelation and subsequent steps of the process (e.g., freeze drying/lyophilization) in this shaped vessel.

In some embodiments of the invention, a cell culture matrix is a defined matrix, e.g., the components of the matrix are known and/or are from a defined source or defined extract, such as alginate. In some embodiments, a cell culture matrix is animal origin-free. In some embodiments, a cell culture matrix is stable at room temperature. In some embodiments of the invention, a cell culture matrix is negatively charged.

In some embodiments of the invention, a cell culture matrix comprises a polysaccharide. In some embodiments, polysaccharides include, but are not limited to, alginates, gellan, gellan gum, xanthan, agar, and carrageenan. In some embodiments, a cell culture matrix comprises 1, 2, 3, 4, 5, 6, 7, 8, 9, 10 or more polysaccharides. Polysaccharide matrices of the invention may be prepared from a polysaccharide solution with or without the addition of a cross-linker. In some embodiments, a cell culture matrix is a polysaccharide sponge, e.g., comprising alginate.

Alginate is typically harvested from the brown seaweed Laminaria hyperborean and is commercially available. Alginates are typically in the form of calcium, magnesium and sodium salts. Alginates have been used in the food, cosmetic and pharmaceutical industry for many years. Alginates are polysaccharides composed of units of mannuronic and guluronic acids, the percentages of each determined by the type and qualities of alginate desired. An example of a brand of alginate that can be used for a cell culture matrix comprising alginate is Pronova MVG UP (e.g., SKU #28023316, Pronova Biopolymer, Drammen, Norway). In some embodiments, an alginate has an apparent viscosity of 300-500 mPa's.

In some embodiments, a polysaccharide matrix of the invention comprises an alginate selected from the group of alginates characterized by having: (i) a mannuronic acid (M) residue content in the range of between about 25% and about 65% of total residues; (ii) a guluronic acid (G) residue content in the range of between about 35% and about 75% of total residues; (iii) a M/G ratio of about 1/3 or about 1.86/1; and (iv) a viscosity of the final alginate solution having 1% w/v alginate, from which the sponge is obtained in the range between about 50 cP to about 800 cP.

In some embodiments, a polysaccharide matrix of the invention comprises a mannuronic acid (M) residue content of between about 20% to about 70%, about 20% to about 60%, about 20% to about 50%, about 20% to about 40%, about 20% to about 30%, about 30% to about 70%, about 40% to about 70%, about 50% to about 70%, about 60% to about 70%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, or about 65% to about 70%, of total residues.

In some embodiments, a polysaccharide matrix of the invention comprises a guluronic acid (G) residue content in the range of between about 30% to about 80% of total residues, about 30% to about 70% of total residues, about 30% to about 60% of total residues, about 30% to about 50% of total residues, about 30% to about 40% of total residues, about 40% to about 80% of total residues, about 50% to about 80% of total residues, about 60% to about 80% of total residues, about 70% to about 80% of total residues, about 40% to about 45% of total residues, about 45% to about 50% of total residues, about 50% to about 55% of total residues, about 55% to about 60% of total residues, about 60% to about 65% of total residues, about 65% to about 70% of total residues, about 70% to about 75% of total residues, about 75% to about 80% of total residues or about 45% to about 65% of total residues.

In some embodiments, a polysaccharide matrix of the invention comprises a M/G ratio of about 1/6 to about 2/1, about 1/3 to about 2/1, about 1/2 to about 2/3, about 5/6 to about 2/1, about 1/1 to about 2/1, about 1.3/1 to about 2/1, about 1.6/1 to about 2/1, about 1/6 to about 1.6/1, about 1/6 to about 1.32/1, about 1/6 to about 1/1, about 1/6 to about 5/6, about 1/6 to about 2/3, about 1/6 to about 1/2, about 1/6 to about 1/3, about 1/3 to about 2/3, about 2/3 to about 1.3/1, or about 1.3/1 to about 1.6/1.

In some embodiments of the invention, a cell culture matrix has at least one characteristic selected from the group consisting of an average pore size in the range between about 1 μm to about 1000 μm; an average distance between the pores being the wall thickness of the pores in the range between about 0.1 μm to about 1000 μm; or an E-modulus of elasticity being a measure of the rigidity of the sponge in the range of between about 1 kPa to about 1000 kPa. In some embodiments of the invention, a cell culture matrix has at least one characteristic selected from the group consisting of an average pore size in the range between from about 10 μm to about 300 μm; an average distance between the pores being the wall thickness of the pores in the range between from about 5 μm to about 270 μm or about 56 μm to about 270 μm; and an E-modulus of elasticity being a measure of the rigidity of the sponge in the range of between from about 50 kPa to about 500 kPa.

In some embodiments, a cell culture matrix will have an average pore size of between from about 1 μm to about 500 μm; about 1 μm to about 250 μm; about 1 μm to about 100 μm; about 1 μm to about 50 μm; about 1 μm to about 25 μm; about 1 μm to about 10 μm; about 1 μm to about 5 μm; about 10 μm to about 1000 μm; about 25 μm to about 1000 μm; about 50 μm to about 1000 μm; about 100 μm to about 1000 μm; about 250 μm to about 1000 μm; about 500 μm to about 1000 μm; about 5 μm to about 25 μm; about 15 μm to about 40 μm; about 25 μm to about 50 μm; about 40 μm to about 75 μm; about 75 μm to about 100 μm; about 100 μm to about 250 μm; or about 250 μm to about 500 μm.

In some embodiments, a cell culture matrix will have an average distance from the pores being the wall thickness of the pores between from about 1 μm to about 1000 μm, about 10 μm to about 1000 μm, about 50 μm to about 1000 μm, about 100 μm to about 1000 μm, about 250 μm to about 1000 μm, about 500 μm to about 1000 μm, about 0.1 μm to about 5 μm, about 0.1 μm to about 1 μm, about 0.1 μm to about 10 μm, about 0.1 μm to about 25 μm, about 0.1 μm to about 50 μm, about 0.1 μm to about 100 μm, about 0.1 μm to about 250 μm, about 0.1 μm to about 500 μm, about 0.1 μm to about 1000 μm, about 1 μm to about 10 μm, about 10 μm to about 25 μm, about 25 μm to about 50 μm, 50 μm to about 100 μm, about 100 μm to about 250 μm, and about 250 μm to about 500 μm.

In some embodiments, a cell culture matrix will have an E-modulus of elasticity being a measure of the rigidity of the sponge between from about 10 kPa to about 1000 kPa, about 50 kPa to about 1000 kPa, about 100 kPa to about 1000 kPa, about 250 kPa to about 1000 kPa, about 500 kPa to about 1000 kPa, about 1 kPa to about 10 kPa, about 1 kPa to about 100 kPa, about 1 kPa to about 250 kPa, about 1 kPa to about 500 kPa, about 10 kPa to about 25 kPa, about 25 kPa to about 50 kPa, about 50 kPa to about 100 kPa, about 100 kPa to about 250 kPa and about 250 kPa to about 500 kPa.

In some embodiments, a cell culture matrix comprising a polysaccharide is produced using a solution of the polysaccharide, wherein the solution is between from about 0.001% to about 10%; 0.01% to about 10%; 0.1% to about 10%; 1% to about 10%; 5% to about 10%; 0.001% to about 1%; 0.001% to about 0.1%; 0.01% to about 0.1%; 0.1% to about 1%; 0.5% to about 1.5%; 1% to about 2%; 2% to about 3%; 3% to about 4%; about 4% to about 5%; about 5% to about 7.5%; or about 7.5% to about 10% polysaccharide by weight/volume. In some embodiments, the cell culture matrix comprises alginate and has at least one of the above described characteristics.

In some embodiments, a cell culture matrix is an alginate matrix or alginate sponge. Alginates are natural polysaccharide polymers. The word “alginate” refers to a family of polyanionic polysaccharide copolymers derived from brown sea algae and comprising 1,4-linked β-D-mannuronic (M) and α-L-guluronic acid (G) residues in varying proportions. Alginates occur naturally as copolymers of D-mannuronate (M) and L-guluronate (G) and have different monomer compositions when isolated from different natural sources. The block length of monomer units, overall composition and molecular weight of the alginate influence its properties. For example, calcium alginates rich in G are stiff materials. Alginate is soluble in aqueous solutions at room temperature and forms stable gels in the presence of certain divalent cations such as calcium, barium, and strontium, as well as in the absence of such cations under certain conditions such as, for example, reduced pH or special processing conditions, e.g., see U.S. Pat. No. 6,425,918. Moreover, alginates are commercially available from a number of manufacturers, e.g., to produce the alginates according to stringent pharmaceutical requirements set by the European and U.S. pharmaceutical regulatory bodies.

Polysaccharide matrices of the invention include, but are not limited to, matrices comprising an alginate derived from brown sea algae selected from the group consisting of alginate Pronatal™ LF 120 (LF 120) derived from Laminaria hyperborea, alginate Pronatal™ LF 20/60 (LF 20/60) derived from Laminaria hyperborea, alginate MVG™ (MVG) derived from Laminaria hyperborea, alginate Pronatal™ HF 120 (HF 120) derived from Laminaria hyperborea, alginate Pronatal™ SF 120 (SF 120) derived from Laminaria hyperborea, alginate Pronatal™ SF 120 RB (SF 120 RB) derived from Laminaria hyperborea, alginate Pronatal™ LF 200 RB (LF 200 RB) derived from Laminaria hyperborea, alginate Manugel™ DMB (DMB) derived from Laminaria hyperborea, Keltone™ HVCR (HVCR) derived from Macrocystis pyrifera, and Keltone™ LV (LV derived from Macrocystis pyrifera.

The porosity and sponge morphology of polysaccharide matrices (e.g., sponges) of the invention may utilize various formulation and processing parameters which may be varied in the process of the invention, and hence it is possible to produce a wide variety of sponges of macroporous nature suitable for cell culture and/or vascularization. In some embodiments of the invention, an alginate matrix is produced using a three step process involving 1) a gelation step in which a polysaccharide solution is gelated in the presence of a cross-linking agent; 2) followed by a freezing step, and 3) finally a drying step, e.g., by lyophilization, to yield a porous sponge. By altering the conditions at each stage, for example, the concentration of a polysaccharide, the presence or absence of a cross-linking agent and the concentration thereof, the shape of a vessel in which the gelation step is carried out, and the rapidity of a freezing step, it is thereby possible to obtain a very broad range of polysaccharide sponges of various shapes, having various pore sizes and distribution and hence also varying mechanical properties. Some embodiments of the invention provide a method for producing a cell culture matrix comprising alginate as shown in FIG. 2.

In some embodiments, a polysaccharide matrix (e.g., comprising alginate) is formulated wherein the polysaccharide is used in the form of a sodium polysaccharide (e.g., alginate) solution having a concentration of polysaccharide between about 1% to about 3% w/v. In some embodiments, a cell culture matrix comprising alginate is formulated wherein the alginate is used in the form of a sodium alginate solution having a concentration of alginate between about 1% to about 3% w/v. to provide an alginate concentration, e.g., between from about 0.1% to about 2% w/v in the final solution from which the matrix (e.g., sponge) is obtained.

In some embodiments of the invention, a polysaccharide matrix may also comprise a cross-linking agent. In some embodiments, a cross-linking agent is selected from the group consisting of the salts of calcium, copper, aluminum, magnesium, strontium, barium, tin, zinc, chromium, organic cations, poly(amino acids), poly(ethyleneimine), poly(vinylamine), poly(allylamine), and polysaccharides. In some embodiments, a cross-linking agent for use in the preparation of the sponges of the invention is selected from the group consisting of calcium chloride (CaCl₂), strontium chloride (SrCl₂) and calcium gluconate (Ca-Gl). In some embodiments, a cross-linker is used in the form of a cross-linker solution having a concentration of cross-linker sufficient to provide a cross-linker concentration between about 0.1% to about 0.3% w/v in the final solution from which the matrix (e.g., sponge) is obtained.

In some embodiments, a crosslinking agent may be any suitable agent with at least two functional groups which are capable of covalently bonding to a carboxylic acid group and/or alcohol group of an alginate or modified groups therefrom. Crosslinking agents of higher functionality may also be used. For example, polyamines such as bifunctional, trifunctional, star polymers or dendritic amines are useful and these can be made, for example, by conversion from corresponding polyols. In some embodiments, crosslinking agents are those with at least two nitrogen-based functional groups such as, for example, diamine or dihydrazide compounds; non-limiting examples thereof being diamino alkanes, Jeffamine series compounds, adipic acid dihydrazide and putrescine. In some embodiments, a crosslinking agent is lysine or an ester thereof, e.g., the methyl or ethyl ester.

Crosslinking can be conducted before, after or simultaneously with the gelling, e.g., by action of the divalent metal cations. For certain applications the crosslinking is conducted either before or simultaneously with gelling by a divalent cation, e.g., so as to prevent problems with diffusion of the crosslinking agent to interior portions of the gelled material.

In some embodiments of the invention, a process for producing a polysaccharide cell culture matrix comprises: (a) providing a polysaccharide solution containing about 1% to about 3% w/v polysaccharide in water; (b) diluting said polysaccharide solution with additional water when desired to obtain a final solution having about 0.5% to about 2% w/v polysaccharide, and subjecting said solution of (a) to gelation, to obtain a polysaccharide gel; (c) freezing the gel of (b); and (d) drying the frozen gel of (c) to obtain a polysaccharide cell culture matrix. In some embodiments, a process further comprises the addition of a cross-linker to said polysaccharide solution of (a), e.g., during the step of gelation (b). In some embodiments, a cross-linker is added in an amount to provide a concentration of cross-linker in the final solution being subjected to gelation of between about 0.1% to about 0.3% w/v. In some embodiments, the gelation step (b) is carried out by intensive stirring of the polysaccharide solution, e.g., in a homogenizer such as at about 31800 RPM for about 3 minutes. In some embodiments, a cross-linker is added to the solution very slowly during intensive stirring of the alginate solution. In some embodiments, the freezing step (c) of the process may be by rapid freezing in a liquid nitrogen bath, e.g., at about −80° C. for about 15 minutes. In some embodiments, the freezing step (c) of the process may be by slow freezing in a freezer, e.g., at about −18° C. for about 8 to 24 hours. In some embodiments, the drying step (d) is by way of lyophilization, e.g., under conditions of about 0.007 mmHg pressure at about −60° C.

Some embodiments of the invention include an alginate sponge prepared from an alginate solution with or without the addition of a cross-linker and wherein said final alginate solution with or without cross-linker from which said sponge is obtained is selected from the group of solutions, having concentrations of alginate or alginate and cross-linker, consisting of: (i) LF 120 alginate about 1% w/v without cross-linker; (ii) LF 120 alginate about 1% w/v and Ca-Gl about 0.1% w/v; (iii) LF 120 alginate about 1% w/v and Ca-Gl about 0.2% w/v; (iv) LF 120 alginate about 1% w/v and SrCl₂ about 0.15% w/v; (v) LF 120 alginate about 1% w/v and CaCl₂ about 0.1% w/v; (vi) LF 120 alginate about 0.5% w/v and Ca-Gl about 0.2% w/v; (vii) LF 20/60 alginate about 1% w/v and Ca-Gl about 0.2% w/v; (viii) HVCR alginate about 0.5% w/v and Ca-Gl about 0.2% w/v; or (ix) HVCR alginate about 1% w/v and Ca-Gl about 0.2% w/v. Some embodiments include sponges obtained from a final solution of LF 120 alginate about 1% w/v and Ca-Gl cross-linker about 0.2% w/v; and a sponge obtained from a final solution of HVCR alginate about 1% w/v and Ca-Gl cross-linker about 0.2% w/v. As examples, alginate matrices are described in U.S. Pat. Nos. 5,885,829, 6,425,918 and 6,642,363.

In some embodiments, a polysaccharide cell culture matrix is formed in a tissue culture vessel. In some embodiments, a tissue culture vessel is coated with a positively charged molecule as described herein.

Some embodiments of the invention provide a method of adhering a cell culture matrix to a surface comprising coating a cell culture matrix with a positively charged molecule. Some embodiments of the invention provide a method of adhering a cell culture matrix to a surface comprising incorporating a positively charged molecule into the matrix. Using alginate sponges as an example, methods for preparing an alginate sponge for cell culture are described, e.g., in U.S. Pat. No. 6,425,918. One of these methods comprises a three step process comprising 1) a gelation step in which a polysaccharide solution is gelated in the presence of a cross-linking agent; 2) followed by a freezing step, and 3) finally a drying step, by lyophilization, to yield a porous sponge. Using this as an example, a positively charged molecule (e.g., polyallylamine (PAA)) may be added before, during or after a gelation step, wherein the positively charged molecule becomes a part of or associated with the cell culture matrix. In some embodiments, this is carried out to adhere or enhance adherence of a cell culture matrix to a negatively charged surface, e.g., a tissue culture vessel comprising polystyrene sulfonic acid.

In some embodiments, a positively charged molecule is coated onto a cell culture matrix. For example a cell culture matrix is contacted with a solution containing a positively charged molecule (e.g., a polyamine such as PAA) for a period of time, e.g., similar to or the same as the coating methods described herein for coating a tissue culture vessel surface. In other words, some of the same or similar parameters (e.g., coating solution concentrations, incubation and/or drying times, etc.) as described herein may be used. In some embodiments, inclusion of a positively charged molecule on and/or into a cell culture matrix can increase the adherence of a cell to the matrix and/or allow the cell to grow on the matrix, e.g., as opposed to growth within the pores of a matrix. Therefore, the invention provides methods for adhering or enhancing adherence of a cell to a cell culture matrix, e.g., utilizing the methods described herein.

If a positively charged molecule is added to during a gelation step, e.g., as discussed herein, the positively charged molecule may be added at a concentration between from about 0.001% to about 40%; about 0.001% to about 0.01%; about 0.1% to about 1%; about 0.01% to about 1%; about 0.01% to about 0.1%; about 0.1% to about 3%; about 1% to about 5%; about 1% to about 2%; about 2% to about 3%; about 3% to about 4%; about 4% to about 5%; about 2% to about 4%; about 5% to about 10%; about 10% to about 20%; or about 20% to about 40% by weight/volume.

In some embodiments, a cell culture matrix comprises a biologically active molecule, e.g., a growth factor, a cell adhesion molecule, an integrin, a cell attachment peptide, a vitamin, an amino acid, a trace element, a peptide growth factor, an enzyme, a proteoglycan or a polysaccharide. In some embodiments, a cell culture matrix (e.g., comprising alginate) comprises an RGD, a YIGSR (SEQ ID NO:1) peptide, a REDV (SEQ ID NO:2) peptide, a GRGDY (SEQ ID NO:3) peptide, a GREDVY (SEQ ID NO:4) peptide (e.g., endothelial cell specific), a RGDS (SEQ ID NO:5) peptide, a LDV peptide, a LRGDN (SEQ ID NO:6) peptide, a PDSGR (SEQ ID NO:7) peptide, a RGDT (SEQ ID NO:8) peptide, a DGEA (SEQ ID NO:9) peptide, and/or a neurite extension sequence (e.g., IKVAV (SEQ ID NO:10)) peptide e.g., see U.S. Pat. No. 6,642,363). In some embodiments, a biologically active molecule is bonded through an uronic acid residue, e.g., on the side chain.

In some embodiments of the invention, a cell culture matrix is optionally sterilized prior to cell culturing. Essentially any sterilization process can be utilized that is compatible with the cell culture matrix and its intended use. For example, the integrity of some cell culture matrices may impacted by certain sterilization techniques, e.g., gamma irradiation at certain doses. Sterilization is an optional step/procedure. For example in some embodiments, cell culture matrices can be produced under sterile conditions, therefore eliminating or reducing contamination to acceptable levels. In some embodiments, cells are cultured in a cell culture matrix in the presence of at least one antibiotic and/or at least one antifungal compound. In some embodiments, a cell culture matrix is sterilized using at least one of the following: irradiation (e.g., gamma or ultraviolet), ethylene oxide sterilization, or electron beam sterilization. In some embodiments, a cell culture matrix is sterilized prior to forming a matrix (e.g., before lyophilization) or after the matrix is formed.

In some embodiments of the invention, a cell culture matrix is sterilized by using a sterilizing gas treatment. In some embodiments of the invention, a cell culture matrix is sterilized by ethylene oxide gas treatment, e.g., using a standard ethylene oxide sterilization apparatus. In some embodiments, cell culture matrices are exposed to about 100% ethylene oxide. In some embodiments, a cell culture matrix is exposed to a sterilization gas (e.g., ethylene oxide) at a relative humidity of about 70%, e.g., for about 3.5 h at, e.g., about 55° C. The samples can then be aerated with warm air flow at atmospheric pressure, e.g., for at least about 48 hours to remove residual ethylene oxide from the alginate sponge. In some embodiments, a cell matrix is sterilized by exposure to ethylene oxide for 24 hr, followed by degassing/aeration for 24 hr. In some embodiments, a cell culture matrix is exposed to a gas containing between about 1% to about 100%, about 10% to about 100%, about 25% to about 100%, about 50% to about 100%, about 60% to about 100%, about 70% to about 100%, about 80% to about 100%, about 85% to about 100%, about 90% to about 100%, about 95% to about 100%, about 98% to about 100%, about 10% to about 25%, about 25% to about 50%, about 50% to about 60%, about 60% to about 70%, about 70% to about 80%, about 80% to about 90%, about 90% to about 95%, about 80% to about 85%, about 85% to about 90% or about 85% to about 95% of a sterilization gas such as ethylene oxide. In some embodiments, the relative humidity during gas sterilization and/or subsequent degassing/aeration is between from about 1% to about 100%, about 25% to about 100%, about 50% to about 100%, about 75% to about 100%, about 1% to about 75%, about 1% to about 50%, about 1% to about 25%, about 10% to about 25%, about 25% to about 50%, about 50% to about 75%, or about 75% to about 100%. In some embodiments, gas sterilization and/or subsequent degassing/aeration takes place for a time between from about 1 minute to about 72 hours, about 1 minute to about 5 minutes, about 5 minutes to about 10 minutes, about 10 minutes to about 30 minutes, about 30 minutes to about 1 hour, about 1 hour to about 1.5 hours, about 1.5 hours to about 2 hours, about 2 hours to about 3 hours, about 3 hours to about 4 hours, about 4 hours to about 6 hours, about 6 hours to about 12 hours, about 12 hours to about 18 hours, about 18 hours to about 24 hours, about 24 hours to about 36 hours, about 36 hours to about 48 hours, about 48 hours to about 60 hours, or about 60 hours to about 72 hours. In some embodiments, gas sterilization and/or subsequent degassing/aeration takes place at a temperature between from about 2° C. to about 10° C., about 10° C. to about 25° C., about 25° C. to about 50° C., about 50° C. to about 75° C., or about 75° C. to about 100° C.

In some embodiments, a cell culture matrix is stored at room temperature, until use. In some embodiments, a cell culture matrix is stored under refrigeration, until use. In some embodiments, a cell culture matrix is stored at a temperature between from about −120° C. to about 37° C., about −85° C. to about 37° C., about −70° C. to about 37° C., about −15° C. to about 37° C., about −5° C. to about 37° C., about 0° C. to about 37° C., about 2° C. to about 37° C., about 4° C. to about 37° C., about 10° C. to about 37° C., about 20° C. to about 37° C., about 30° C. to about 37° C., −120° C. to about 30° C., −120° C. to about 25° C., −120° C. to about 10° C., −120° C. to about 0° C., −120° C. to about −10° C., −120° C. to about −50° C., about −90° C. to about −50° C., about −50° C. to about −30° C., about −30° C. to about −15° C., about −15° C. to about 5° C., about −5° C. to about 10° C., about 0° C. to about 5° C., about 0° C. to about 10° C., about 2° C. to about 15° C., about 10° C. to about 20° C., about 20° C. to about 30° C. or about 30° C. to about 40° C. In some embodiments, a cell culture matrix is stored in bags (e.g., sealed or laminated).

### Adherence of 3-D Cell Culture Matrices to Tissue Culture Vessels and Their Surfaces

One problem with some cell culture matrices is that they become detached from a surface, e.g., when hydrated. This can lead to several disadvantages such as a detached/floating cell culture matrix can interfere with pipetting. For example, it is advantageous to have 96 well tissue culture plates containing cell culture matrices for high throughput assays. In some cases, if cell culture matrices become detached and rise to the top of the cell culture medium, the matrices can interfere with the pipetting or aspirating of the cell culture medium, e.g., by clogging the pipette tips. This can lead to variable results and/or slow down what is preferred to be a high through put process. Additionally, some cells growing in a detached matrix, may exhibit different characteristics as compared to non-detached matrix. Also matrices that detach may allow cells to adhere to and grow in a 2D manner on the surface of the tissue culture vessel (e.g., polystyrene) where the matrix had been in contact, resulting in a 3D/2D cell culture.

While performing feasibility studies for producing and using cell culture matrices the inventors observed that in some cases, the matrices became detached from cell culture vessel surfaces. Not wishing to be bound by theory, the inventors believe that at least main two factors may be responsible for the detachment: 1) the cell culture matrix comprises gas (e.g., bubbles) which contribute to floating and lifting of the matrix from the surface (e.g., a surface of a tissue culture vessel) and/or 2) the interaction/attraction between the matrix and the surface of the vessel is weak, non-existent or even repelling (e.g., due to similar charges). Inter alia, the present invention provides methods to mitigate both of these effects.

Therefore, the present invention provides methods of adhering a cell culture matrix (e.g., a 3-D cell culture matrix) to a surface, such as a surface on a tissue culture vessel. Additionally, the present invention provides methods of decreasing, inhibiting and/or preventing the detachment of a cell culture matrix from a surface, such as a surface on a tissue culture vessel. The present invention also provides methods of attracting, enhancing or creating an attraction of one surface to another surface.

The present invention also provides methods for decreasing, inhibiting and/or preventing the amount of gas in or formed in a cell culture matrix. Methods are also provided that decrease, inhibit and/or prevent a cell culture matrix from floating, e.g., when rehydrated or after rehydration (e.g., in a cell culture medium). Methods are also provided that decrease, inhibit and/or prevent a cell culture matrix from detaching from a surface, e.g., due at least in part to “floating” caused by the presence of gas in the matrix. When referring to gas in a cell culture matrix, the gas can come from, inter alia, gas (e.g., air) left after pipetting or hydration of a matrix and/or gas produced in the matrix, e.g., by cells in the matrix and/or a chemical reaction within a matrix.

Some methods of the invention include providing a cell culture matrix and contacting the matrix with cells (e.g., a suspension of cells). In some embodiments, the cells are a plurality of embryonic stems cells suspended in a solution of cell culture medium.

Some embodiments of the invention provide a method of adhering a charged (e.g., negatively charged) first surface to a like charged (e.g., negatively charged) second surface. Some methods of the invention comprise: (a) coating one of the surfaces with a charged (e.g., negative or positive) molecule and (b) contacting the first surface with the second surface. Some embodiments of the invention provide a method of adhering a substrate to a negatively charged and/or hydrophobic first surface wherein the method comprises: (a) coating one of the surfaces with a positively charged molecule and (b) contacting the substrate with the second surface.

Some embodiments of the invention provide a method of producing a cell culture matrix. Some methods comprise: (a) coating a first surface with a charged molecule (e.g., positive or negative) and (b) contacting the first surface with the cell culture matrix. In some embodiments, the charged molecule has a charge opposite of the charge of the cell culture matrix.

Some embodiments of the invention provide a method of culturing cells on a cell culture matrix. Some of these methods comprise: (a) coating a first surface with a charged molecule (e.g., opposite charge of the cell culture matrix); (b) contacting the first surface with the cell culture matrix; and (c) contacting the cells with the cell culture matrix under conditions suitable for culturing the cells.

Some embodiments of the invention provide a method of determining an effect of at least one compound on a cell comprising: (a) coating a first surface with a positively charged molecule; (b) contacting the first surface with the cell culture matrix; (c) contacting the cells with the cell culture matrix under conditions suitable for culturing the cells; (d) contacting the cells of (c) with the at least one compound; and (e) determining the effect or lack of effect on the cell. It is understood the contacting the cells with a cell culture matrix does not necessarily mean that the cells attach or grow on the matrix. For example, the cells can grow in the matrix, such as in the pores of a matrix as spheroids.

Some embodiments of the invention provide a method of adhering a cell culture matrix to a surface comprising coating a cell culture matrix with a positively charged molecule. Some embodiments of the invention provide a method of adhering a cell culture matrix to a surface comprising incorporating a positively charged molecule into and/or onto the matrix, e.g., as described herein.

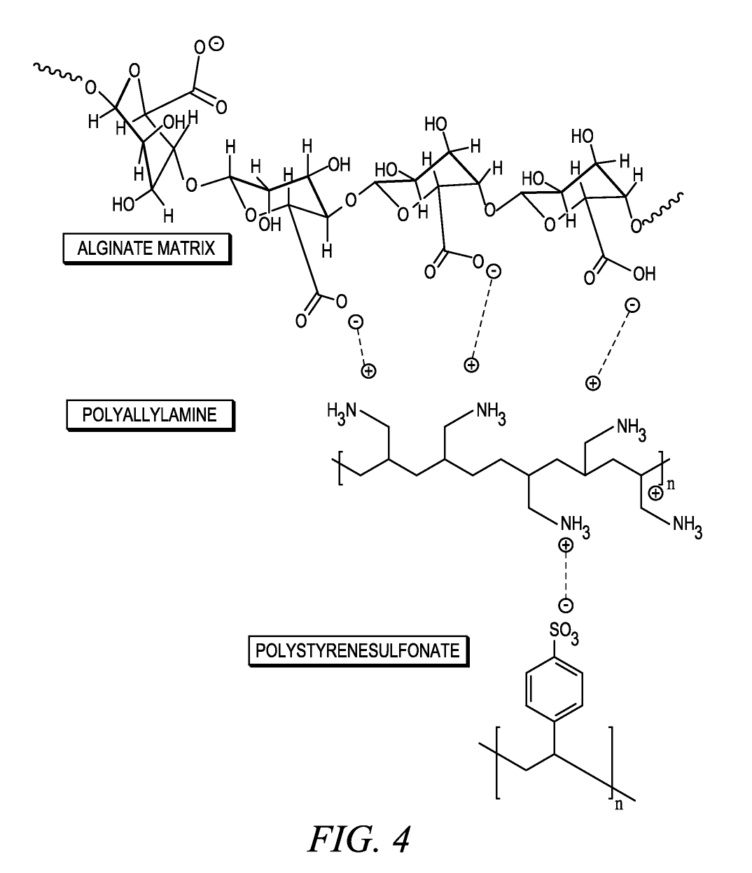

Some tissue culture vessels comprise polystyrene sulfonate (or polystyrene sulfonic acid). Polystyrene sulfonate is a type of polymer and ionomer based on polystyrene. It may be prepared by polymerization or copolymerization of sodium styrene sulfonate or by sulfonation of polystyrene. A cell culture vessel and/or surface comprising polystyrene sulfonate will typically exhibit a negative charge. Therefore, a cell culture matrix with a neutral charge or especially a negative charge may not adhere well to a cell culture vessel comprised of polystyrene sulfonate. FIG. 4 shows as an example of proposed interactions between a polystyrene sulfonate surface, a polyallylamine and a matrix comprising alginate. Thus, the present invention provides methods of adhering a negatively or neutral charged first surface with a surface comprising polystyrene sulfonate and or comprising polypropylene.

In some embodiments, a surface comprises a positively charged molecule, such as a polyamine, e.g., a tissue culture vessel surface or a surface of a cell culture matrix. In some embodiments, a positively charged molecule is coated onto a surface.

In some embodiments of the invention, a solution comprising a positively charged molecule is contacted with a surface for a period of time to coat the surface. In some embodiments, after this period of time the solution is removed. In some embodiments, after the solution is removed the surface is allowed to dry for a period of time. In some embodiments, the surface is washed or rinsed with another solution (e.g., water) to remove and/or reduce the amount of positively charged molecule not bound to the surface. In some embodiments, the surface can be rinsed/washed and/or dried multiple times.

In some embodiments, a surface is contacted with a positively charged molecule for a period of time between from about 1 second to about 1 week, about 1 second to about 6 days, about 1 second to about 5 days, about 1 second to about 4 days, about 1 second to about 72 hours, about 1 second to about 60 hours, about 1 second to about 48 hours, about 1 second to about 36 hours, about 1 second to about 24 hours, about 1 second to about 20 hours, about 1 second to about 16 hours, about 1 second to about 12 hours, about 1 second to about 10 hours, about 1 second to about 8 hours, about 1 second to about 6 hours, about 1 second to about 4 hours, about 1 second to about 2 hours, about 1 second to about 1 hour, about 1 second to about 45 minutes, about 1 second to about 30 minutes, about 1 second to about 15 minutes, about 1 second to about 10 minutes, about 1 second to about 5 minutes, about 1 second to about 1 minute, about 1 minute to about 5 minutes, about 5 minutes to about 10 minutes, about 10 minutes to about 15 minutes, about 15 minutes to about 20 minutes, about 20 minutes to about 30 minutes, about 30 minutes to about 45 minutes, about 45 minutes to about 60 minutes, about 60 minutes to about 90 minutes, about 90 minutes to about 120 minutes, about 1 hour to about 3 hours, about 2 hours to about 5 hours, about 2 hours to about 7 hours, about 5 hours to about 10 hours, about 10 hours to about 15 hours, about 15 hours to about 20 hours, about 20 hours to about 23 hours, about 20 hours to about 30 hours, about 1 day to about 2 days, about 2 day to about 3 days, about 3 day to about 4 days, about 4 day to about 7 days, about 1 minute to about 36 hours, about 1 minute to about 60 minutes, about 15 minutes to about 45 minutes, about 0.5 hours to about 1.5 hours, about 1 hour to about 2 hours, about 1 minute to about 16 hours, about 1 minute to about 12 hours, about 1 minute to about 5 hours, about 1 minute to about 2 hours, about 1 hour to about 5 hours, or about 5 hours to about 12 hours.

In some embodiments, subsequent to contact or coating with a positively charged molecule, a surface is allowed to dry for a period of time between from about 1 minute to about 72 hours; about 1 minute to about 48 hours; about 1 minute to about 36 hours; about 1 minute to about 24 hours; about 1 minute to about 12 hours; about 1 hour to about 16 hours; about 1 hour to about 6 hours; about 1 hour to about 3 hours; about 12 hours to about 24 hours; about 24 hours to about 36 hours; about 36 hours to about 48 hours; about 48 hours to about 72 hours or more. In some embodiments, the solution containing a positively charged molecule comprises a positively charged molecule (e.g., a polyamine) between from about 0.001% to about 40%; about 0.001% to about 0.01%; about 0.1% to about 1%; about 0.01% to about 1%; about 0.01% to about 0.1%; about 0.1% to about 3%; about 1% to about 5%; about 5% to about 10%; about 10% to about 20%; or about 20% to about 40% by weight/volume.

Typically during a coating process, a solvent containing a positively charged molecule is contacted with a surface for a period of time and then removed. In some embodiments, it may be important to remove the unbound positively charged molecule leaving little or no amount of unbound molecules. In some embodiments, a solvent comprising a positively charged molecule is removed from a first surface using aspiration or pipetting. In some embodiments, a solvent comprising a positively charged molecule is removed from a first surface using multiple aspirations and/or multiple pipetting. In some embodiments, a coating process also involves a “wash” step to assist with the removal of unbound positively charged molecules. A wash step can involve, for example, contacting a coated surface with a solution that does not contain (or contains very low concentrations as compared to the coating solution) a positively charged molecule. This “wash solution” can be, for example, water or a buffered solution. Typically, a wash solution is selected so as not to interfere, inhibit or have detrimental effects with regards to the coating process and/or the intended use. For example, typically one would select a wash solution with a pH that does not cause the release of a significant number or percentage of positively charged molecules from the coated surface. However, in some cases it may be desirable to remove some positively charged molecules from the surface, so in this case a wash solution may be designed to release positively charged molecules from the coated surface.

Depending on the desired end use of the matrix, the amount of positively charged molecule used or remaining (e.g., after coating) may need to be adjusted or optimized. For example, in some cases, excessive positive charge (e.g., above a certain concentration) may be toxic to certain cells. In these cases, one can optimize the amount of positively charged molecule to balance with the toxic effects for the desired end use. For example, depending on the end use, some toxicity may be acceptable and/or a percentage of “detached” matrices may be acceptable. For example, if 10 replicates are desired and 50% of the matrices detach, then 20 or more matrices per condition can be tested, which should result in at least 10 non-detached matrix replicates.

Some embodiments of the invention provide methods and compositions related to adhering a first surface to a second surface wherein the surfaces are each hydrophobic or hydrophilic or wherein one is hydrophobic and the other is hydrophilic. For example, some embodiments of the invention comprise coating a first surface (e.g., a hydrophilic first surface) with a hydrophobic molecule and contacting the first surface with a second hydrophobic surface, e.g., wherein the contacting is performed in the presence of a liquid, such as an aqueous liquid such as water. In some embodiments, the hydrophobic molecule is incorporated into a surface. In some embodiments, a first surface is hydrophobic and is then coated with a hydrophilic molecule and a second surface is hydrophilic. In some embodiments, adhering two surfaces can utilize a combination of methods described herein relating to both the hydrophobicity/hydrophilicity and the charge of the surfaces.

In some embodiments of the invention, a tissue culture vessel is a tissue culture plate selected from the group consisting of a 6-well plate, an 8-well plate, a 12-well plate, a 24-well plate, a 48-well plate, a 60-well plate, a 72-well plate, a 98-well plate, a 384-well plate and a 1536-well plate. In some embodiments of the invention, a tissue culture vessel is a tissue culture flask selected from the group consisting of a 25 cm² flask, a 75 cm² flask, a 92.6 cm² flask, a 100 cm² flask, a 150 cm² flask, a 162 cm² flask, a 175 cm² flask, a 225 cm² flask, and a 235 cm² flask. In some embodiments, a tissue culture vessel is a tissue culture tissue culture dish (e.g., round)

Some embodiments of the invention provide methods of preparing a cell culture matrix and shipping the cell culture matrix (e.g., to a customer). Methods of the invention can be utilized to increase adherence of a cell culture matrix to a surface. Some embodiments of the invention decrease the tendency of a cell culture matrix to detach from a surface, e.g., during shipping such as commercial shipping by another party.

### Adherence of a Cell to a Surface

Some cell types or even clones of the same parental cell do not bind well to a typical tissue culture surface. In some instances, cell culture surfaces can be coated with a molecule(s) (e.g., a polylysine) that enhances binding of a cell. This enhancement of adherence can allow cells to be cultured that can not be cultured or are not cultured as efficiently on an uncoated surface. Additionally, enhanced adherence of a cell can be advantageous in methods involving manipulation of cells, such as involving high throughput screening.

The invention additionally provides methods of adhering a cell to a surface comprising coating a surface with a positively charged molecule (e.g., a polyamine) and contacting the surface with a cell. Some embodiments include culturing the cell while the cell is contacted with the coated surface. In some embodiments, a cell is contacted with the coated surface in serum-free conditions. Some embodiments comprise contacting the cell with the coated surface under conditions suitable for culturing the cell. In some embodiments of the invention, a positively charged molecule is a polyallylamine. In some embodiments of the invention, a positively charged molecule is not a polylysine. In some embodiments, a surface is coated with a positively charged molecule, e.g., as described herein, and then contacted with a cell. In some embodiments, a surface is at least a portion of a tissue culture vessel. In some embodiments of the invention, the positively charged molecule is polyallylamine. In some embodiments of the invention, polyallylamine is used to coat a tissue culture vessel for growing a cell that can be grown on or is typically grown on a polylysine coated surface. In some embodiments, polyallylamine can be used in place of polylysine in cell culturing applications.

In some embodiments, a cell has greater adherence to the coated surface as compared to the uncoated surface. Methods for determining and evaluating a cell's level of adherence to a surface are known in the art, e.g., see U.S. patent application Ser. No. 10/805,536. For example, increased adherence to a surface (e.g., a tissue culture support) can be determined using various cell-washing protocols. For example, cells can be grown on a surface of a tissue culture plate and then exposed to an automatic plate washer using predetermined wash settings and the amount of cells still attached after the washing can be compared to a control tissue culture plate with the same cells and exposed to the same wash setting. In some embodiments, washing is done manually, e.g., by pipetting and not using an automatic washer.

In some embodiments of the invention, adherence is tested by plating cells on treated/coated and untreated/uncoated 24-well tissue culture plates and allowing the cells to adhere, e.g., overnight. Cells are then treated as follows: cells are washed with (e.g., 1 ml D-PBS (no Ca⁺⁺ no Mg⁺⁺) (D-PBS)), incubated in (e.g., 250 μl) Versene (e.g., 1:5000 (Invitrogen) for 5 minutes), the Versene is removed and (e.g., 250 μl) trypsin is added, e.g., for 1 minute, and removed. Cells are incubated with D-PBS for 10 minutes. Cells are washed with D-PBS. The D-PBS is removed and cells are incubated in (e.g., 250 μl) trypsin, e.g., for 1 or 2 minutes. Following the above treatments, the cells are stained, e.g., with 0.05% Crystal Violet (CV) in PBS+10% Formalin and then rinsed. The amount of stain (e.g., CV) in the treated/coated wells is compared with the untreated/uncoated wells.

### Positively Charged Molecules

Essentially any positively charged molecule can be utilized in the invention. In some embodiments of the invention, any positively molecule can be utilized that does not completely inhibit or have significantly detrimental effects on the intended use. A compound could have some detrimental effects, but still be useful for the intended purpose. Using cell culture matrices as an example, positively charged molecules for use in the present invention include any that are or can be adapted to be compatible with the culture of cells. For example, their presence is not toxic to the cell or the toxicity is at a level that does not completely interfere with the purpose for culturing the cells.

In some embodiments of the invention, a positively charged molecule is a polymer. In some embodiments, a polymer is a homopolymer or a copolymer. In some embodiments, a positively charged molecule is a monomer. In some embodiments, both a polymer and monomer are used as positively charged molecules, e.g., both are coated on a surface.

In some embodiments, a positively charged molecule is a polyamine. Polyamines are organic compounds having two or more primary amino groups—such as putrescine, polyallylamine, cadaverine, spermidine, and spermine. Amines are organic compounds and a type of functional group that contains nitrogen as the key atom. In some instances, polyamines typically have cations that are found at regularly-spaced intervals, unlike, e.g., Mg++ or Ca++, which are point charges.

In some embodiments of the invention, polyamine polymers have primary amine groups, secondary amine groups, tertiary amine groups, quaternary ammonium groups, and/or mixtures thereof. Examples of polyamines include, but are not limited to, a polyvinylamine (e.g., Polyvinylamine HCl), a polybutylamine, a polyisobutylamine, a polyallylamine, a polyethyleneimine, a polyalkyleneamine, a polyazetidine, a polyvinylguanidine, a poly(DADMAC) (i.e., a poly(diallyl dimethyl ammonium chloride), a cationic polyacrylamide, a polyamine functionalized polyacrylate, and mixtures thereof.

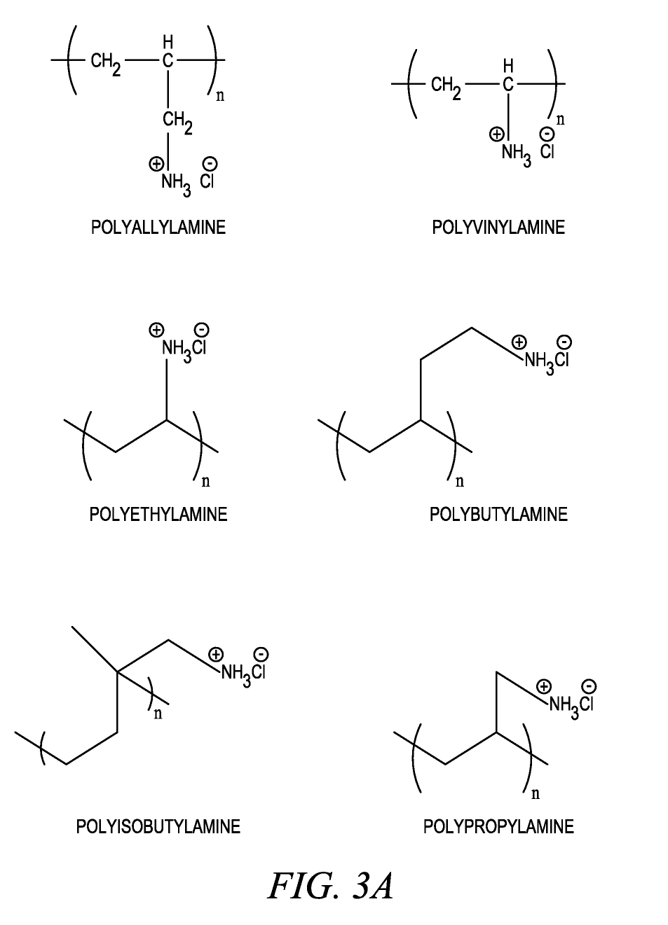

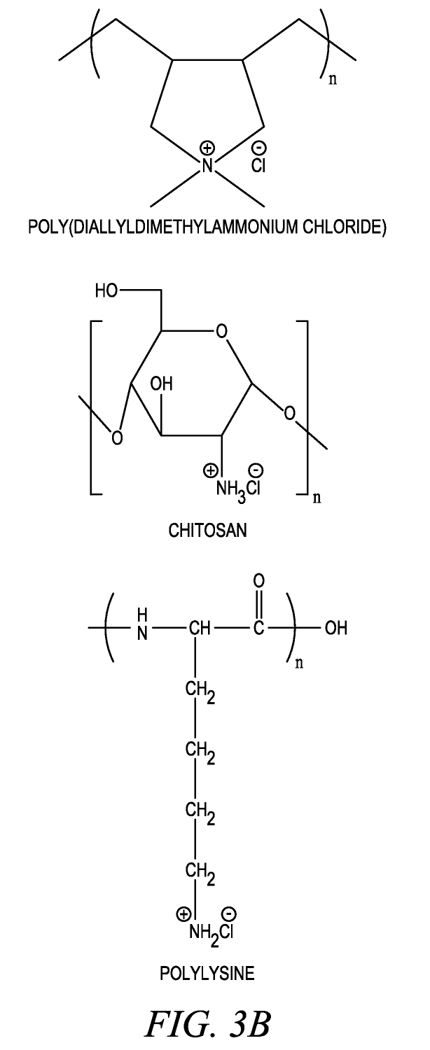

Structures of examples of positively charged molecules that can be utilized in the invention are shown in FIG. 3.

Allylamine (also known as 3-aminopropene, 3-aminopropylene, monoallylamine, 2-propenamine, 2-propen-1-amine, or allyl amine) is an organic amine with the molecular formula C₃H₇N and is an example of a positively charged molecule or polyamine that can be used in the present invention.

In some embodiments, a positively charged molecule comprises vinylamine. As an example, some embodiments of the invention can use a homopolymers and/or copolymer of vinylamine, such as copolymers of vinylformamide and comonomers for example, which are converted to vinylamine copolymers. Comonomers can be any monomer capable of copolymerizing with vinylformamide. Nonlimiting examples of such monomers include, but are not limited to, acrylamide, methacrylamide, methacrylonitrile, vinylacetate, vinylpropionate, styrene, ethylene, propylene, N-vinylpyrrolidone, N-vinylcaprolactam, N-vinylimidazole, monomers containing a sulfonate or phosphonate group, vinylglycol, acrylamido(methacrylamido)alkylene trialkyl ammonium salt, diallyl dialkylammonium salt, C₁₋₄alkyl vinyl ethers such as methyl vinyl ether, ethyl vinyl ether, isopropyl vinyl ether, n-propyl vinyl ether, t-butyl vinyl ether, N-substituted alkyl (meth)acrylamides substituted by a C₁₋₄alkyl group as, for example, N-methylacrylamide, N-isopropylacrylamide, and N,N-dimethylacrylamide, C₁₋₂₀alkyl(meth)acrylic acid esters such as methyl methacrylate, ethyl methacrylate, propyl acrylate, butyl acrylate, hydroxyethyl acrylate, hydroxyethyl methacrylate, hydroxypropyl acrylate, hydroxypropyl methacrylate, hydroxybutyl acrylate, hydroxybutyl methacrylate, 2-methylbutyl acrylate, 3-methylbutyl acrylate, 3-pentyl acrylate, neopentyl acrylate, 2-methylpentyl acrylate, hexyl acrylate, cyclohexyl acrylate, 2-ethylhexyl acrylate, phenyl acrylate, heptyl acrylate, benzyl acrylate, tolyl acrylate, octyl acrylate, 2-octyl acrylate, nonyl acrylate, and octyl methacrylate. Specific copolymers of polyvinylamine include, but are not limited to, copolymers of N-vinylformamide and vinyl acetate, vinyl propionate, a C₁₋₄alkyl vinyl ether, a (meth)acrylic acid ester, acrylonitrile, acrylamide and vinylpyrrolidone.

The positively charged molecules of the invention can be in different forms or coated using different forms (e.g., salts). Using polyallylamine as an example, a polyallylamine can be an AcOH, CF₃COOH, CCl₃COOH, or CH₃SO₃H form. In some embodiments, a positively charge molecule, such as polyallylamine, is an aliphatic or aromatic acid salt.

In some embodiments of the invention a positively charged molecule is in the D-form. In some embodiments of the invention a positively charged molecule is in the L-form. In some embodiments of the invention a positively charged molecule is a mixture or the D-form and the L-form.

In some embodiments of the invention, a positively charged molecule (e.g., a polyamine such as polyallylamine) has a molecular weight of between from about 5,000 to about 1,000,000, about 5,000 to about 10,000, about 5,000 to about 15,000, about 5,000 to about 50,000, about 5,000 to about 100,000, about 5,000 to about 500,000, about 20,000 to about 300,000, about 500,000 to about 1,000,000, about 250,000 to about 1,000,000, about 100,000 to about 1,000,000, about 50,000 to about 1,000,000, about 25,000 to about 50,000, about 50,000 to about 75,000, about 65,000 to about 70,000, about 75,000 to about 100,000, about 100,000 to about 250,000, about 100,000 to about 300,000, about 250,000 to about 500,000, about 70,000 to about 150,000, or about 150,000 to about 300,000. In some embodiments, a positively charged molecule is PLL >300,000 (e.g., Sigma-Aldrich catalog# P1524); PLL 70,000-150,000 (e.g., Sigma-Aldrich catalog# P1274); PLL 150,000-300,000 (e.g., Sigma-Aldrich catalog# P1399); PEI 10,000 (e.g., Sigma-Aldrich catalog# 408727); PAA 15,000 (e.g., Sigma-Aldrich catalog# 283125); or PAA 70,000 (e.g., Sigma-Aldrich catalog# 283223).

In some embodiments, a positively charged molecule (e.g., a polyamine) has at least two or a plurality of nitrogen atoms per molecule. In some embodiments, a positively charged molecule (e.g., a polyamine) has between from about 2 to about 10,000, about 2 to about 5,000, about 2 to about 1,000, about 2 to about 600, about 2 to about 300, about 2 to about 100, about 2 to about 50, about 100 to about 10,000, about 200 to about 10,000, about 500 to about 10,000, about 1,000 to about 10,000, about 5,000 to about 10,000, about 2 to about 100, about 100 to about 250, about 200 to about 300, about 250 to about 500, about 500 to about 600, about 600 to about 800, about 800 to about 1,000, about 1,000 to about 1,200, about 1,200 to about 1,600, about 1,600 to about 2,000, about 2,000 to about 2,500, about 2,500 to about 3,500, about 3,500 to about 4,500, about 4,500 to about 5,500, about 4,600 to about 4,700, about 5,500 to about 6,500, about 6,500 to about 7,500, about 7,500 to about 8,000, about 7,500 to about 8,500, about 8,500 to about 9,000 or about 9,000 to about 10,000 nitrogen atoms per molecule. In some embodiments, a positively charged molecule (e.g., a polyamine) has about 262, about 542, about 1084, about 1162, about 1226, about 2324, about 4648, or about 7746 nitrogen atoms per molecule.

In some embodiments, coating of a first surface comprises contacting the first surface with a positively charged molecule in a solvent. In some embodiments, a solvent is water, an alcohol, or a glycol. In some embodiments, a glycol is methanol, ethanol, ethylene glycol, propylene glycol, or mixtures thereof. In some embodiments, a positively charged molecule is present in a solvent at a percent by weight in the solvent of from about 0.0001% to about 99%, about 0.0001% to about 75%, about 0.0001% to about 50%, about 0.0001% to about 40%, about 0.0001% to about 30%, about 0.0001% to about 20%, about 0.0001% to about 10%, about 0.0001% to about 1%, about 0.0001% to about 0.1%, about 0.0001% to about 0.01%, about 0.0001% to about 0.001%, about 0.001% to about 0.01%, about 0.01% to about 0.1%, about 0.1% to about 1%, about 1% to about 2%, about 1% to about 3%, about 1% to about 5%, about 3% to about 7%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 60%, about 60% to about 70%, about 70% to about 80%, about 80% to about 90%, or about 90% to about 99%.

In some embodiments of the invention, a positively charged molecule, such as a polyamine, is crosslinked, e.g., before or after coating a surface with the positively charged molecule. In some embodiments using a polyamine, a second coating solution which contains an optional inorganic salt having a polyvalent cation (e.g., a cation having a valence of two, three, or four) can be applied to a surface with a polyamine or to a surface comprising a polyamine. In some embodiments, a polyvalent cation (e.g., metal cation) is capable of interacting (e.g., forming ionic crosslinks) with the nitrogen atoms of the polyamine. In some embodiments, a polyvalent cation can interact (e.g., form ionic links) with the polyamine because of a low pH of the base polymer particles. In some aspects of the invention, an optional inorganic salt applied to surfaces of the base polymer particles has a sufficient water solubility such that polyvalent metal cations are available to interact with the nitrogen atoms of the polyamine. In some embodiments, a polyvalent metal cation of the optional inorganic salt has a valence of +2, +3, +4 or in the range of +2 to +4 and can be, but is not limited to, Mg²⁺, Ca²⁺, Al³⁺, Sc³⁺, Ti⁴⁺, Mn²⁺, Fe²⁺, Fe³⁺, Co²⁺, Ni²⁺, Cu^{+/2+}, Zn²⁺, y³⁺, Zr⁴⁺, La³⁺, Ce⁴⁺, Hf⁴⁺, Au³⁺, and mixtures thereof. In some embodiments, the cations are selected from Mg²⁺, Ca²⁺, Al³⁺, Ti⁴⁺, Zr⁴⁺, La³⁺, and mixtures thereof. In some embodiments, cations are Al³⁺, Ti⁴⁺, Zr⁴⁺, or mixtures thereof. An anion of an inorganic salt is not limited, as long as the inorganic salt has sufficient solubility in water. Examples of anions include, but are not limited to, chloride, bromide, nitrate and sulfate. In some embodiments (e.g., related to cell culture), the polyvalent cations and/or inorganic salt should not have significantly detrimental effects on the intended use

In some embodiments of the invention, a positively charged molecule is a synthetic polyelectrolyte. In some embodiments, a synthetic polyelectrolyte comprises a quaternary ammonium group, such as poly(diallyldimethylammonium chloride) (PDADMA), poly(vinylbenzyltrimethylammonium) (PVBTA), ionenes, poly(acryloxyethyltrimethyl ammonium chloride), poly(methacryloxy(2-hydroxy)propyltrimethyl ammonium chloride), and copolymers thereof. In some embodiments, a synthetic polyelectrolyte comprises a pyridinium group such as poly(N-methylvinylpyridinium) (PMVP), including poly(N-methyl-2-vinylpyridinium) (PM2VP), other poly(N-alkylvinylpyridines), and copolymers thereof. In some embodiments, a synthetic polyelectrolyte comprises protonated polyamines such as poly(allylaminehydrochloride) and polyethyleneimine (PEI).

### Related Methods and “Downstream” Applications

The cell culture matrices of the invention can be utilized for growing a variety of cells. In some embodiments, cells are selected from the group consisting of gingival submucosal cells, dental pulp tissue cell, dentin tissue cells, cementum tissue cells, periodontal tissue cells, oral submucosa tissue cells, tongue tissue cells, plant cells, prokaryotic cell, eukaryotic cells, mammalian cells, vertebrate cells, mouse cells, human cells, hybridoma cells, hepatocytes, fibroblast cells, stem cells, embryonic stem cells, hematopoietic stem cells, bone marrow cells, muscle cells, cardiac cells, keratinocytes, cancer cells, tumor cells and tumor cell lines, prostate cells, brain cells, neurons, endothelial cells, CHO cells, 293 cells, and PerC.6 cells and cell lines derived from each of these cell types. In some embodiments, cells can be primary cells or cell lines.

Uses of cell culture matrices as described herein include use for in vitro, in vivo or ex vivo, including but not limited to, in vitro culturing of plant cells and algae; the delivery to a tissue or organ of genetically engineered viral vectors, non-viral vectors, polymeric microspheres or liposomes (e.g., encoding and/or containing a therapeutic agent for said tissue or organ); in vitro fertilization of mammalian oocytes; storage of fertilized mammalian oocytes, or other mammalian cells cultured in vitro; the storage of plant cells or algae cultured in vitro; and the transplantation of cells grown on or within a cell culture matrix in vitro into a tissue of a patient, e.g., in need of the cells as a result of tissue damage, removal or dysfunction.

Some embodiments of the invention provide a cell culture matrix (e.g., an alginate sponge) for use as a matrix, substrate or scaffold for growing mammalian cells in vitro. In some embodiments, a cell culture matrix of the invention is used as a matrix, substrate or scaffold for implantation into a patient to replace or repair tissue that has been removed or damaged. Some cell culture matrices can be use as an implanted support for therapeutic drug delivery into a desired tissue, the drug delivery being by way of the action of genetically engineered cells or natural cells carried by a matrix and expressing therapeutic drugs, the cells expressing the drug or expressing regulatory proteins to direct the production of the drug endogenously in the tissue. In some embodiments, a therapeutic drug expressed by cells carried on or in the matrix is a therapeutic protein wherein the cells express the protein or express regulatory proteins to direct the production of the protein endogenously in the tissue into which the matrix is implanted.

Once cells are introduced or contacted with a cell culture matrix of the invention and/or the desired experimental parameters met (culture duration, spheroid formation, inducers, etc.), assays and/or experiments can be performed as desired.

In some embodiments, a cell culture matrix of the invention can be used to assess cell viability and proliferation, e.g., assessments are performed after exposing the cells to various conditions. In some embodiments, viability and proliferation assessment is conducted using, e.g., Alamar Blue™ (e.g. catalog# DAL1100, Invitrogen, Carlsbad, Calif.), directly on cells and/or spheroids within the matrix.

In some embodiments, assays or experiments are performed on spheroids or cells isolated from the matrix, e.g., a matrix comprising alginate. In some embodiments, a matrix or polysaccharide containing matrix (e.g. alginate matrix) is dissolved with trisodium citrate, e.g., iso-osmolar. As an example, about 55 mM trisodium citrate (e.g., about 4 ml) is added to a 15 ml centrifuge tube containing sponges/matrices (e.g., 5) from wells of a 96-well plate(s). The tube is then inverted, e.g., about 1-2 minutes at room temperature The tube can be centrifuged, e.g., about 7 minutes at about 400 xg, and supernatant removed. In some embodiments, Versene (e.g., 10 ml) is added to a centrifuge tube containing sponges/matrices (e.g., 5), and placed on a hematology inverter at about 37° C. for about 20 minutes. After these manipulations, one can proceed with an assay or a desired experimentation. To be clear, some embodiments of the invention also provide assay or experiments that are performed directly on the cells in/on the cell culture matrix.

Some embodiments of the invention provide a method of isolating spheroids and/or cells from a cell culture matrix (e.g., comprising alginate). In some embodiments, methods comprise using a trisodium citrate solution to isolate spheroids or cells. In some embodiments, a trisodium citrate solution is made iso-osmolar in comparison to the growth medium. Osmolarity can be measured with an osmometer and adjusted using standard procedures, e.g., adding 1 g/L NaCl to a solution will typically raise the osmolarity by 30 mOsm.

Some embodiments of the invention provide a method of isolating individual cells. In some embodiments, individual cells are isolated from an isolated spheroid, e.g., as described herein. For example, then TrypLE™ Select (e.g., about 2 ml, catalog# 12563-011, Invitrogen) or Trypsin-EDTA is added to in a 15 ml centrifuge tube with spheroids, placed at about 37° C. and triturate (pipette up and down) several times over about 15-20 minutes. After dissolution of spheroids, add about 10 ml of growth medium or buffer, spin about 7 minutes at about 400 xg, and remove supernatant. Then proceed with assay or desired experimentation.

Some embodiments of the invention provide methods for processing and/or staining cells. Some embodiments provide a method comprising culturing cells in a cell culture matrix, embedding the matrix containing spheroids in paraffin according to standard protocols. The embedded cells can be processed using standard procedures, e.g., sectioned, fixed and stained.

### 7. EXAMPLES

The invention is now described with reference to the following examples. These examples are provided for the purpose of illustration only and the invention should in no way be construed as being limited to these examples but rather should be construed to encompass any and all variations which become evident as a result of the teachings provided herein.

Whereas, particular embodiments of the invention have been described herein for purposes of description, it will be appreciated by those skilled in the art that numerous variations of the details may be made without departing from the invention as described in the appended claims.

### Example 1

### Preliminary Experiments to Evaluate Gas in a Cell Culture Matrix

The following experiment utilizes cell culture matrices comprising alginate as an example. These methods can be applicable to other cell culture matrices, e.g., other cell culture matrices comprising a polysaccharide

The inventors noticed upon hydration of an alginate matrix that there were white opaque floaters. These white opaque floaters were noticed on several random lots and were thought to be caused by increased air bubbles or gas in the matrix. Initially, the following variables were evaluated to determine their influence on the number of these floaters: 1) calcium to alginate ratio; 2) absolute concentration of alginate; 3) freeze dryer shelf location; 4) use of the pouch; and 5) homogenizer cleaning before use.

1) The calcium to alginate ratio refers to the weight ratio of calcium gluconate to sodium alginate. A ratio of 0.0718 implies 0.067 g of calcium gluconate to be used with 0.933 g of sodium alginate. The calcium to alginate ratio directly relates to the amount of cross-linking of the hydrogel. 2) The absolute concentration of alginate refers to the weight percent of alginate in the gel, generally in the range of 1%. As the % alginate increases, so does the “firmness” of the hydrogel. 3) Freeze drier shelf location refers to either the upper, middle or lower locations within the freeze drier. 4) Original lyophilization of alginate sponges were done with the plates not being wrapped in any kind of packaging. It was considered that enclosing the sponge plates in a pouch even with one end open for evacuation of moisture may cause white opaque floaters. 5) The homogenizer was rinsed with water and finally 70% alcohol to eliminate alginate from the previous run and maintain an aseptic process. This alcohol was not dried, although it was flushed, but it could have been contributing to white opaque floaters.

Cell matrices were produced in 96 well plates as described in Example 7, except for the variables measured herein (e.g., Table 1) and the calcium to alginate ratio was 0.081 (instead of 0.0718) and the cell culture plates were uncoated, meaning no PAA was used. The cell matrices were then hydrated in the 96 well plates using a medium of equi-mixtures of [Williams Medium E, DMEM, DMEM/F12 and Waymouth's MB 752/1]+10% FBS. The matrices were then observed at 1 and 24 hours after hydration. The results are shown in Table 2. It appears that the calcium to alginate ratio is an important factor for these conditions (Table 2).

A second experiment was performed to more precisely define the optimal alginate concentration and the calcium to alginate ratio to eliminate white, opaque floaters. The results are shown in Table 3. These results show that various calcium/alginate ratios decrease the amount of floaters. For example a calcium/alginate ratio of 0.0718 (0.067/0.933) may be utilized. However, other ratios can be used to give satisfactory results.

TABLE 1

| | | Number | | | Freeze | | |
|---|---|---|---|---|---|---|---|
| | Number | of plates | | | dryer | with or | |
| | of plates | retained | Alginate | | shelf | without | Homogenizer |
| Plate I.D | sent to GI | in NZ | Conc. | Ratio | location | pouch | cleaned? |
| A1 | 4 | 1 | 0.80% | 0.075/0.925 | Low | Pouch | Yes |
| A2 | 4 | 1 | 0.80% | 0.075/0.925 | High | Pouch | Yes |
| A3 | 6 | 1 | 0.80% | 0.075/0.925 | High | — | Yes |
| A4 | 4 | 1 | 0.80% | 0.075/0.925 | Low | — | Yes |
| B5 | 4 | 1 | 0.80% | 0.075/0.925 | Low | Pouch | No |
| B6 | 4 | 1 | 0.80% | 0.075/0.925 | High | Pouch | No |
| B7 | 5 | 1 | 0.80% | 0.075/0.925 | High | — | No |
| B8 | 4 | 1 | 0.80% | 0.075/0.925 | Low | — | No |
| C9 | 4 | 1 | 1.20% | 0.075/0.925 | Low | Pouch | Yes |
| C10 | 4 | 1 | 1.20% | 0.075/0.925 | High | Pouch | Yes |
| C11 | 6 | 1 | 1.20% | 0.075/0.925 | High | — | Yes |
| C12 | 4 | 1 | 1.20% | 0.075/0.925 | Low | — | Yes |
| D13 | 4 | 1 | 1.20% | 0.075/0.925 | Low | Pouch | No |
| D14 | 4 | 1 | 1.20% | 0.075/0.925 | High | Pouch | No |
| D15 | 5 | 1 | 1.20% | 0.075/0.925 | High | — | No |
| D16 | 4 | 1 | 1.20% | 0.075/0.925 | Low | — | No |
| E17 | 4 | 1 | 1.00% | 0.06/0.940 | Low | Pouch | Yes |
| E18 | 4 | 1 | 1.00% | 0.06/0.940 | High | Pouch | Yes |
| E19 | 4 | 1 | 1.00% | 0.06/0.940 | High | — | Yes |
| E20 | 3 | 1 | 1.00% | 0.06/0.940 | Low | — | Yes |
| F21 | 4 | 1 | 1.00% | 0.06/0.940 | Low | Pouch | No |
| F22 | 4 | 1 | 1.00% | 0.06/0.940 | High | Pouch | No |
| F23 | 4 | 1 | 1.00% | 0.06/0.940 | High | — | No |
| F24 | 4 | 1 | 1.00% | 0.06/0.940 | Low | — | No |
| G25 | 3 | 1 | 1.00% | 0.075/0.925 | Low | Pouch | Yes |
| G26 | 3 | 1 | 1.00% | 0.075/0.925 | High | Pouch | Yes |
| G27 | 4 | 1 | 1.00% | 0.075/0.925 | High | — | Yes |
| G28 | 4 | 1 | 1.00% | 0.075/0.925 | Low | — | Yes |
| H29 | 4 | 1 | 1.00% | 0.075/0.925 | Low | Pouch | No |
| H30 | 4 | 1 | 1.00% | 0.075/0.925 | High | Pouch | No |
| H31 | 3 | 1 | 1.00% | 0.075/0.925 | High | — | No |
| H32 | 4 | 1 | 1.00% | 0.075/0.925 | Low | — | No |
| I33 | 4 | 1 | 1.00% | 0.09/0.910 | Low | Pouch | Yes |
| I34 | 3 | 1 | 1.00% | 0.09/0.910 | High | Pouch | Yes |
| I35 | 4 | 1 | 1.00% | 0.09/0.910 | High | — | Yes |
| I36 | 4 | 1 | 1.00% | 0.09/0.910 | Low | — | Yes |
| J37 | 4 | 1 | 1.00% | 0.09/0.910 | Low | Pouch | No |
| J38 | 4 | 1 | 1.00% | 0.09/0.910 | High | Pouch | No |
| J39 | 4 | 1 | 1.00% | 0.09/0.910 | High | — | No |
| J40 | 4 | 1 | 1.00% | 0.09/0.910 | Low | — | No |

TABLE 2

| | Hydration results after 1 hour | | | | Hydration results after 24 hours | | | |
|---|---|---|---|---|---|---|---|---|
| | Normal | | | | Normal | | | |
| | translucent | | Opaque | Opaque | translucent | | Opaque | Opaque |
| I.D | sponges | Floaters | sinkers | floaters | sponges | Floaters | sinkers | floaters |
| A1 | — | — | 4 | 92 | — | — | — | 96 |
| A2 | — | — | 1 | 95 | — | — | — | 96 |
| A3 | — | — | — | 80 | — | — | — | 80 |
| A4 | — | — | 16 | 80 | — | — | — | 96 |
| B5 | 88 | — | — | — | 86 | 2 | — | — |
| B6 | 26 | 47 | — | — | 1 | 72+ | — | — |
| B7 | 63 | 19 | — | — | 38 | 44 | — | — |
| B8 | 93 | 3 | | | 17 | 79+ | — | — |
| C9 | 96 | — | — | — | 89 | 7 | — | — |
| C10 | 95 | 1 | — | — | 88 | 8 | — | — |
| C11 | 56 | — | — | — | 45 | 11 | — | — |
| C12 | 93 | 3 | — | — | 87 | 9 | — | — |
| D13 | 96 | — | — | — | 96 | — | — | — |
| D14 | 96 | — | — | — | 95 | 1 | — | — |
| D15 | 96 | — | — | — | 95 | 1 | — | — |
| D16 | 96 | — | — | — | 96 | — | — | — |
| E17 | 96 | — | — | — | 96* | — | — | — |
| E18 | 96 | — | — | — | 96* | — | — | — |
| E19 | 96 | — | — | — | 96* | — | — | — |
| E20 | 96 | — | — | — | 96* | — | — | — |
| F21 | 96 | — | — | — | 96* | — | — | — |
| F22 | 96 | — | — | — | 94* | 2* | — | — |
| F23 | 96 | — | — | — | 96* | — | — | — |
| F24 | 96 | — | — | — | 96* | — | — | — |
| G25 | 36+ | 60+ | — | — | 21+ | 75+ | — | — |
| G26 | 15+ | 81+ | — | — | 15+ | 81+ | — | — |
| G27 | — | — | 77+ | 19+ | 36+ | 60+ | — | — |
| G28 | 4 | — | 54+ | 18+ | 4+ | 68+ | — | — |
| H29 | 33+ | 63+ | — | — | 16+ | 80+ | — | — |
| H30 | 62+ | 34+ | — | — | 49+ | 47+ | — | — |
| H31 | 87+ | 4+ | — | 5+ | 86+ | 5+ | — | 5+ |
| H32 | 74+ | 22+ | — | — | 53+ | 43+ | — | — |
| I33 | — | — | — | 96 | — | — | — | 96 |
| I34 | — | — | — | 96 | — | — | — | 96 |
| I35 | — | — | 2 | 93 | — | — | — | 95 |
| I36 | — | — | 2 | 93 | — | — | — | 95 |
| J37 | — | — | 34 | 62 | — | — | 3 | 93 |
| J38 | — | — | 3 | 93 | — | — | — | 96 |
| J39 | 1 | — | 11 | 84 | 1 | — | 11 | 84 |
| J40 | 61 | 10 | — | 25 | 53 | 18 | — | 25 |

+Sponges are semi opaque/translucent.
*Sponges started to dissolve after 24 hours.
Plates A3, B5, B7, C11, and H32 were partially thawed before loading into the lyophilizer.

TABLE 3

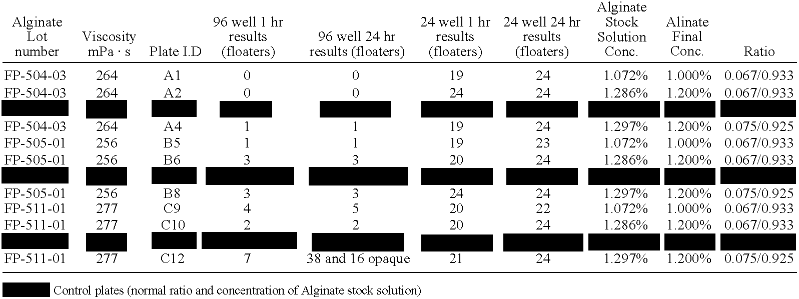

### Example 2

### Evaluating Various Methods for Adhering a Cell Culture Matrix

In addition to the white opaque floaters (as discussed in Example 1), other matrices, while not white opaque and floating, would be translucent and non-attached over part or most of the sponge surface, so sponges would extend up into the well off of the polystyrene surface when prepared as described in Example 7. Variable percentages of sponges from 10-60% would be involved in this, in 96 well cell culture plates. For 24 well cell culture plates, essentially all sponges would become mostly non-attached and exist somewhere in the medium. So the phenomenon was more frequent and pronounced in the larger well sizes. Various technologies to keep the sponges on the polystyrene surface were investigated.

First a model was developed to indicate a relative value of various technologies which could then be tested for use with the matrices. A cell culture matrix “time to float” assay was optimized where a cell culture matrix was formed in a 96 well cell culture plate. The matrices were hydrated, removed from the wells and then placed in a well of a 24 well plate that had been treated with different technologies. Media was then added against the side of the well to overlay the sponge and the time to float was visually observed and indicated. Using this assay, several options for adhering the cell culture matrices to the bottom of a well in 24 well tissue culture plate (BD Falcon #353047) were investigated.

The following different surfaces and/or treatments were evaluated: Corning UV Universal Bind (# 2504) and Corning Carbo-Bind (# 2508), use of transwells (Corning # 3422), Bio-Glue (Loctite Corporation, Rocky Hill, Conn., #4011), Starwells plates (Lockwell Star, Polysorb # 448-496), different formulations of trays (polypropylene (Costar # 29442-064), high binding (BD Falcon # 353047), low binding (Costar Corning # 3473), glass), and poly-D-lysine (PDL, BD Falcon # 354414). The PLL coating was prepared by dissolving PLL into distilled water to make a 0.1% solution which was then membrane-filtered at 0.2 um. Next 100 ul of PLL solution was added to each well of a 96 well tray and incubated for 30 minutes at room temperature. After this, all of the PLL was pipetted out of each well. (Rinsing was not performed). The trays were then placed under a laminar flow hood with lids on overnight to allow PLL to dry.

None of the different surfaces and/or treatments worked better than non-treated control wells except poly-L-lysine (PLL) coating where an improvement was noted.

### Example 3

### Evaluation of Different Compounds for Enhancing Adherence

Several positively charged compounds were evaluated for their ability to adhere or enhance adherence of a cell culture matrix to a surface, e.g., of a tissue culture plate. In this experiment PLL, poly(ethyleneimine) (PEI), and poly (allylamine hydrochloride) (PAA) were evaluated. PLL 70-150K (Catalog# P1274, Sigma, St. Louis, Mo.), PLL 150-300K (Catalog# P1399, Sigma) and PLL >300K (Catalog# P1524, Sigma) were each tested at 0.1% and 0.01% w/v. PEI 10K (Catalog#408727, Sigma) was tested at 1.0%, 0.1% and 0.01% w/v. PAA 15K (Catalog# 283125, Sigma) and PAA 70K (Catalog# 283223, Sigma) were each tested at 1.0%, 0.1% and 0.01% w/v. Two control plates were evaluated that had no coating.

For preparation of the plates, 100 μl of solution was added to each well of a 96 well plate (Grenier Cellstar plates) and incubated at room temperature for 30 minutes. After this, the solution was totally withdrawn (no washes) and dried with lids off under a laminar flow hood about 1 hour. Two control plates were used that were not coated. Sponge formation was as per Example 7.

The alginate cell culture matrices were hydrated and the wells were observed for detachment of the matrix. Since it is more difficult to observe the inner wells of a 96-well plate, two different methods for observation were used. One method (Top/Bottom) was to view all 96 wells of a plate from the top and bottom of the plate to observe detachment. The second method was to view from the side the outer wells along the perimeter of the 96-well plate for detachment, which assesses 36 out of 96 wells which is a sampling rate of 37.5%.

Results are shown in Table 4. Of the parameters tested, poly(allylamine hydrochloride) 70K at 1% seemed best at preventing the matrices from detaching from the polystyrene surface of the 96-well plate. However, all of the parameters tested (except for may be PEI, 10K, 0.01%) showed increased adherence as compared to controls.

TABLE 4

| | 1 Hour | | 24 Hour | |
|---|---|---|---|---|
| | Top/Bottom | Side | Top/Bottom | Side |
| PLL, 70-150K, 0.1% | 7 | 5 | 6 | 9 |
| PLL, 70-150K, 0.01% | 0 | 7 | 4 | 8 |
| PLL, 150-300K, 0.1% | 4 | 1 | 7 | 5 |
| PLL, 150-300K, | 12 | 9 | 16 | 11 |
| PLL, >300K, 0.1% | 12 | 13 | 20 | 16 |
| PLL, >300K, 0.01% | 3 | 13 | 7 | 16 |
| PEI, 10K, 1.0% | 1 | 5 | 3 | 5 |
| PEI, 10K, 0.1% | 11 | 14 | 18 | 16 |
| PEI, 10K, 0.01% | 29 | 24 | 40 | 31 |
| PAA, 15K, 1.0% | 1 | 0 | 1 | 0 |
| PAA, 15K, 0.1% | 9 | 5 | 13 | 7 |
| PAA, 15K, 0.01% | 12 | 7 | 15 | 8 |
| PAA, 70K, 1.0% | 0 | 0 | 0 | 0 |
| PAA, 70K, 0.1% | 10 | 7 | 1 | 8 |
| PAA, 70K, 0.01% | 13 | 9 | 22 | 13 |
| Control plate 1 | 29 | 18 | 53 | 30 |
| Control plate 2 | 23 | 20 | 43 | 31 |

### Example 4

### Evaluating PAA

During experiments with PAA using 96-well plates a significant percentage of star-shaped, crenated matrices with a “yellowing” of the medium were observed when the medium was added. In addition, aberrant Alamar Blue results were observed. A PAA small scale validation run in 96-well plates presented with debris and fewer spheroids that was not seen in wells without PAA. Lack of total withdraw of PAA was suspected. In other words, excess PAA remained in the wells. Visual and microscopic assessment of trays from a PAA validation run showed obvious abnormalities. All the control plates without PAA had normal numbers of spheroids with total absence of brown wispy debris. All of the PAA plates showed yellow wells at culture initiation, abnormally low numbers of spheroids and/or significant amounts of brown wispy debris.

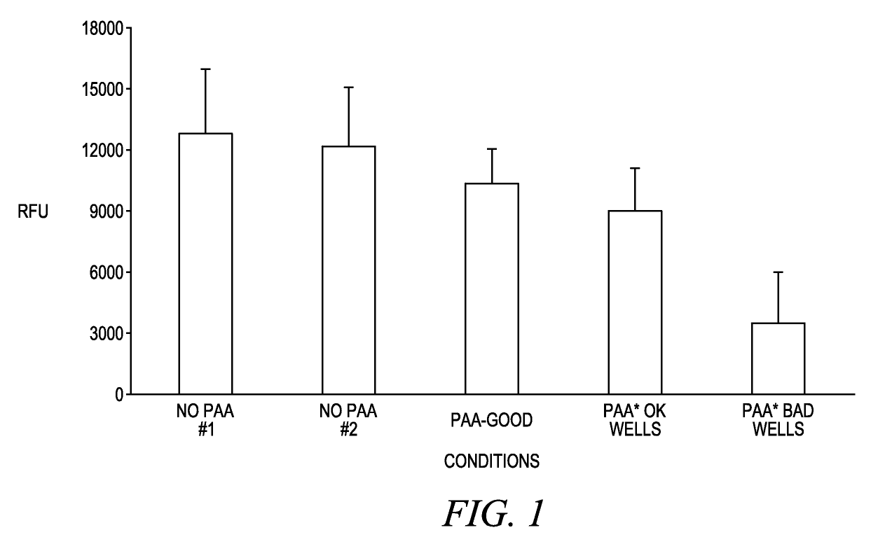

FIG. 1 shows a small scale PAA trial. These results indicated that decreases in Alamar Blue RFU's result with increasing levels of remaining PAA. (PAA-good=no noticeable yellowing; PAA*OK=wells with barely discernable yellowing; and PAA*Bad=wells with obvious yellowing of medium post-reconstitution).

The Alamar blue assay determines the metabolic reducing potential within a cell culture, the greater the potential (greater the number of viable cells) the greater the reduction of Alamar blue to yield a fluorescent compound which is measured on a fluorescence plate reader. Alamar blue reagent: Biosource, # DAL1100, Invitrogen, Inc.

In another experiment, similar results were obtained, see Table 5. Nine PAA-coated plates showed similar results as above with the same level of stringency of PAA removal performed. In this experiment, even un-inoculated sponges appeared shriveled in the wells. In addition, again, upon close examination, large numbers of wells became yellow (acidic) upon hydration and inoculation with medium. Interestingly, floating sponges, the reason for using PAA adsorption were essentially eliminated. (Table 5) This confirmed that more stringency is needed in removal of excess PAA.

Therefore, an experiment was performed to evaluate more judicious removal of PAA after coating. This involved a technique for removal of excess PAA where the plates are held at 45° and well is “ringed” consecutively around the bottom, one well after the other and repeated 3 times. Subsequent drying involves leaving the plates with lid on under the laminar flow hood overnight. Plates appear dry the next morning. Minor “yellowing” was seen with acceptable spheroid formation and acceptable Alamar blue results. (Table 6) Overtime the yellowed wells appeared normal. (Table 6) This procedure resulted in substantially eliminating floating sponges and appears to have eliminated issues related to excess PAA.

TABLE 5

| Plate | Floating 1 hr | Floating 24 hr | Yellow medium change |
|---|---|---|---|
| No. | (per 96 wells) | (per 96 wells) | (per 96 wells) |
| 1* | 0 | 0 | 40 |
| 2* | 2 | 2 | 46 |
| 3* | 0 | 0 | 43 |
| 4 | 0 | 0 | 18 |
| 5 | 0 | 0 | 14 |
| 6 | 0 | 0 | 0 |
| 7 | 0 | 0 | 5 |
| 8 | 0 | 0 | 25 |
| 9 | 0 | nd | 0 |

*indicates visual shriveled sponges in plate

TABLE 6

| | 1 hour | | 24 hour | |
|---|---|---|---|---|
| | Partial | PAA left-over | Partial | PAA left-over |
| Plate | Detachment | yellow medium | Detachment | yellow medium |
| 1 | 0 | 10 | 0 | 0 |
| 2 | 2 | 4 | 2 | 0 |
| 3 | 0 | 6 | 0 | 0 |
| 4 | 3 | 6 | 3 | 0 |
| 5 | 0 | 0 | 0 | 0 |
| 6 | 0 | 0 | 0 | 0 |
| 7 | 0 | 3 | 0 | 0 |
| 8 | 0 | 3 | 0 | 0 |
| 9 | 0 | 6 | 0 | 0 |
| 10 | 0 | 10 | 0 | 0 |

### Example 5

### Example Using an Optimized PAA-Removal Protocol

While past experiments used a single channel pipettor, this time an eight channel unit was used. The problem with single channel units is that, with many plates to aspirate, it is very difficult to give the proper level of PAA removal stringency to each of the 96 wells for one plate, let alone for 150 plates. Basic removal protocol is as described in Example 4, but now with using an 8 channel pipettor.

The plates are assayed by the 1) hydration test, 2) spheroid test and 3) Alamar blue assay.

Hydration test: 30 ul of medium containing cells (25,000 HepG2-C3A human hepatocarcinoma cells in 30 ul) is added to the sponges, followed by centrifugation at 100 g for 4 minutes. Plates are then incubated at 37° C. for 10 minutes followed by the addition of 200 ul of C3A Mix medium (equal mixture of [DMEM+Waymouth's MB 752/1+William's Medium E+DMEM/F-12]+10% Fetal Bovine Serum. Plates are incubated at 37° C. for 1 hour and observed for presence of floating sponges.

Spheroid test: Incubation is continued for 5 days at 37° C. Then plates are microscopically observed for the formation of spheroids (multicellular spheres of growing cells bonded together) in each of the wells and a count taken of the number of wells with spheroids. It is desired to the all wells have spheroids.

Alamar Blue Assay: At this point 20 ul of Alamar blue reagent is added to each well. Plates are incubated for 30 minutes at 37° C. The relative fluorescence units of each well are now measured with excitation set at 560 nm and emission at 590 nm. For this experiment an RFU of ≧5553 was considered to be acceptable.

A run using this optimized PAA-removal protocol yielded results where all sponges remain attached (no white opaque floaters and no translucent non-attached sponges were observed), there is no yellow media discoloration at cell inoculation and there is an absence of the wispy brown debris. In addition, spheroid formation is exemplary with acceptable Alamar Blue readings. (Table 7) A few sponges were “lifting”, but at the end of the culture this “lifting” could not be observed (sponges were all on well bottoms and appeared high-quality). A control sponge from a previous lot showed some brown wispy debris in several of the wells.

TABLE 7

| | | | | Post |
|---|---|---|---|---|
| | 1 hour hydration | Alamar Blue | Spheroids | culture |
| Plate | (# floaters/96 wells) | (RFU) | (>95% pass) | sponge |
| 1 | 0 | 6213 | 24/24+ | All bottom |
| | | | Many beautiful | |
| 2 | 0 (1 lifting) | 7411 | 24/24+ | All bottom |
| | | | Many beautiful | |
| 3 | 0 | 6951 | 24/24+ | All bottom |
| | | | Many beautiful | |
| 4 | 0 | 7748 | 24/24+ | All bottom |
| | | | Many beautiful | |
| 5 | 0 (1 lifting) | 7659 | 24/24+ | All bottom |
| | | | Many beautiful | |
| 6 | 0 (1 lifting) | 9023 | 24/24+ | All bottom |
| | | | Many beautiful | |
| 7 | 0 (2 lifting) | 7769 | 24/24+ | All bottom |
| | | | Many beautiful | |
| 8 | 0 (1 lifting) | 9030 | 24/24+ | All bottom |
| | | | Many beautiful | |
| 9 | 0 | 7819 | 24/24+ | All bottom |
| | | | Many beautiful | |
| 10 | 0 (2 lifting) | 9476 | 24/24+ | All bottom |
| | | | Many beautiful | |
| Control | | 7461 | 24/24+ | All bottom |
| | | | Many beautiful | |
| | | | Also, brown | |
| | | | wispy debris | |
| | | | in wells. | |

### Example 6

### Shelf-Life Testing

Both real time and accelerated shelf data are presented. Accelerated shelf life was tested according to American Society for Testing and Materials (ASTM) F1980, Accelerated Aging of Sterile Medical Device Packages. A temperature of 45° C. was used (Arrhenius reaction (10° C.=2× chemical rate change)). At 45° C., 1 week simulates about 1 month at 21° C.

A shelf life testing of an alginate matrix using PAA coating of a 96 well plates was performed on alginate matrices produced as described in Example 7.

The plates were incubated at room temperature for 22 days and then at 45° C. for 42 days. Seven days at 45° C. equals 30 days at room temperature, therefore 42 days at 45° C. equals 180 days plus the previous 22 days at room temperature equals the equivalent of 202 days or 6.7 months at room temperature. Results are shown in Table 8 which indicate a first time point shelf life of at least 6.7 months.

TABLE 8

| Condition | Spheroid Formation | Alamar Blue |
|---|---|---|
| PAA @ 45° C. | 24 out of 24 wells | 8254; SD1267 |
| No Coating@ 45° C. | 24 out of 24 wells | 11738; SD4026 |
| Fresh - PAA coating | 24 out of 24 wells | 7991; SD2143 |
| Fresh - no coating | 24 out of 24 wells | 9581; SD3183 |

A subsequent experiment supported a real time shelf life of 4 months and a shelf life of 14.9 months considering accelerated shelf life testing data for PAA coated plates with an alginate matrix prepared as described in Example 7. In these samples, hydration, spheroid formation and Alamar Blue toxicity were well within acceptable ranges.

### Example 7

### Procedure for Producing Alginate Cell Culture Matrices Using PAA

This Example describes an example of a procedure for producing an alginate cell culture matrix in a PAA coated 96 well plate.

Scope: This procedure takes place in a Clean Room Environment, in a Laminar Flow Unit as well as in Fairfax, Freeze drying facility.

Definitions: PAA—Poly(allylamine) hydrochloride; mw—Molecular Weight; WFM—Water For Manufacture; IPA—Isopryl Alcohol

The process of manufacturing these plates takes a minimum of 7 days. A summary of process flow is as follow:

Day 1—Pre-coat 96 well plates with 100 uL of PAA and prepare Calcium D Gluconate Stock solution.

Day 2—Filter Calcium stock to 0.22 um and prepare and filter Alginate stock to 0.22 um.

Day 3—Cross link Alginate stock solution with Calcium stock solution; dispense 100 ul into each well of every plate; package plates in irradiated pouches and chill overnight.

Day 4—Transfer plates to −20° C. freezer for a minimum of 8 hours.

Day 5—Transfer plates to pre-frozen shelves in Lyophilizer.

Day 6—Plates drying in Lyophilizer for minimum of 46 hours at −20° C.

Day 7—Unload dryer and transfer plates to clean room for packaging.

Procedure

Day 1—PAA coating

Ensure all equipment is clean and/or autoclaved.

Using a suitable sized glass beaker, make up the required volume of PAA-Poly(allylamine hydrochloride), mw=70K, Sigma cat.# 283223.

To calculate the volume required, determine how many plates are to be manufactured. Number of plates to be coated×11 ml=volume of PAA

Make up a 1% solution. For example, to coat 120 plates requires 1320 ml of PAA solution. 13.2 g of PAA must be dissolved in 1320 ml of WFM.

Mix the solution until fully dissolved using a magnetic stirrer and bar.

Filter the solution using a suitable sized Stericup (Table 9) filter in a Laminar Flow Hood.

TABLE 9

| Name of Filter | MFG Item number |
|---|---|
| 0.22um GP express PLUS | SCGPU05RE |
| Membrane 500 ml or 1000 ml | SCGPU11RE |

Dispense 100 uL into each well of the plate in a Laminar Flow Hood using filtered tips, e.g., on a multistepper. (Greiner 96 well plates; e.g., 655180 (Individual plates); 655182 (10 pack plates).

Replace the plate lids and leave in the laminar flow for a minimum 30 minutes.

It is possible to dispense all the plates before processing to the aspiration step. Although it is recommended that the plates are aspirated as soon as possible after completion.

Remove the solution using a Vacusafe Comfort Aspirator (item number:158310, supplier John Morris Scientific, NZ. Manufacturer: Integra Biosciences) with stainless steel probe handset. The handset should be autoclaved before use.

First aspiration. The plate is flat on the laminar Flow bench. The multichannel aspirating head is dunked into each well, slightly off center and towards the edge of the well.

Second Aspiration The plate must be held at 45° so that the PM solution collects in the corner of the wells. The head does a 360° sweep twice round the well.

Third Aspiration The plate remains at 45° and the head does a sweep of the bottom of the well where the liquid may remain.

Complete 100% inspection on all the wells after the three aspirations to ensure all the liquid has been removed.

Replace the lid and stack plates in the laminar flow until dry, at least 24 hours. The plates can be left to dry for up to 7 days.

Day 1—Alginate Matrix Preparation

Using a suitable sized glass beaker, make up the required volume of Calcium D-Gluconate Stock solution (Sigma-G4625). To calculate the required volume, determine how many plates are to be dispensed. At least 10 ml of Alginate cross linked solution is needed per plate. Ratio of Calcium to Alginate is 0.067:0.933

A=Number of plates required×12 ml (includes 2 ml overage)

Volume of Calcium D-Gluconate Stock solution needed C=A×0.067.

The concentration of the solution is 2.0%.

To calculate the amount of Calcium D-Gluconate (g) required to make the Calcium D-Gluconate Stock solution=C/100×2.

To make the stock solution, Calcium D-Gluconate is added to warm (30-35° C.) WFM (volume=C). Add the Calcium D-Gluconate slowly to warm WFM (between 30-35° C.). Ensure the WFM is mixing at high speed, on a magnetic stirrer, whilst the Calcium is being added. Do not use the heat option on the stirrer.

Mix at a sufficient speed to ensure the vortex is almost touching the magnetic flea. Cover the beaker with 2 layers of parafilm and continue stirring until the Calcium D-Gluconate has dissolved. This typically takes approximately 18 hours.

Day 2

Using a suitable sized glass beaker, make up the required volume of Sodium Alginate Stock solution (Sodium Alginate Pronova UP MVG NovaMatrix-28023316).

To calculate the required volume, determine how many plates are to be dispensed. At least 10 ml of Alginate cross linked solution is needed per plate. Ratio of Calcium to Alginate is 0.067:0.933

A=Number of plates required×12 ml (includes 2 ml overage).

Volume of Alginate Stock solution needed (B)=A×0.933.

The concentration of the solution should be 1.286%.

To calculate the amount of Sodium Alginate (g) required to make (B) the Alginate Stock solution=B/100×1.286

Measure the exact volume of WFM (volume=B) using a calibrated measuring cylinder.

Add WFM to suitably sized glass beaker and mix using an overhead stirrer on high speed.

Mix at a sufficient speed to ensure the vortex is over half way down the beaker.

Weigh the exact amount of Sodium Alginate required for the stock solution using weighing paper.

Add the Alginate to the stirring WFM slowly.

Cover the solution, where possible, and continue to mix until fully dissolved.

Do not use heat to aid dissolution.

This typically takes less than 2 hours.

Once the Alginate and Calcium stock solution have dissolved, filter both the solutions in a laminar flow using a suitable sized Stericup Filter. (Table 9)

The Alginate stock solution filters very slowly—approximately 500 mls in 45 minutes.

Once filtered the alginate stock solutions can be kept for up to 4 days at 2-8° C. The Calcium stock solution can be kept for up to 3 weeks at 2-8° C.

Day 3

Take both filtered stock solutions to a laminar flow for homogenization.

To ensure the correct amount of Calcium is added to the Alginate it is necessary to confirm the exact volumes of the Alginate stock solution after filtration.

Using a calibrated, autoclaved, glass measuring cylinder measure the volume of Alginate stock solution.

If necessary use a stripette and pipette boy for smaller volumes.

Divide the stock into equal sized portions.

Ensure that each portion is no more than 700 ml.

Calculate the volume of Calcium required to crosslink with each portion of Alginate solution. Ratio of Calcium to Alginate is 0.067:0.933.

Portion of Alginate stock solution=Cross linked volume 0.933

Cross linked volume×0.067=Amount of Calcium required.

Repeat calculation for all portions of Alginate stock solution. Calculate to 1 decimal place.

Calculate the length of time each portion of Alginate stock solution needs to be homogenized.

Volume of Alginate stock solution=Length of time 31.7 (This calculation is a factor)

Place the container holding the Sodium Alginate stock solution in an ice slurry.

If the volume of cross linked solution is greater than 700 mL, attach a calibrated temperature indicator strip to the outside of the container to ensure the temperature does not reach any more than 37° C.

Whilst using the Homogenizer (Heidolph Homogenizer with 18F tool attachment; 595-06000-00-2 (Homogenizer); 596-18010-00-0 (18F tool)) at full speed (26,000 rpm), slowly add the calculated calcium stock to the Alginate Stock.

Add the calcium at a steady rate, approximately 2-3 ml per minute.

All the Calcium stock solution must be added to the Alginate stock solution within the calculated time.

Repeat with all other portions of Alginate Stock Solutions.

Combine all the portions of cross linked Alginate in one sterile container and homogenize for 10 minutes. If using an open beaker, ensure an autoclaved stainless cover plate is used.

Ensure the container is in an ice slurry during homogenization.

Leave the cross linked solution to rest in the laminar flow for at least one hour to allow as many bubbles to be released as possible. Ensure metal cover plate is used, if applicable.

Wipe the homogenizer tool clean, wash using hot water and autoclave.

Obtain the correct quantity of coated or uncoated plates.

Slowly dispense 100 ul into each of the 96 wells using filtered pipette tips. The Alginate solution is very viscous, after dispensing 100 ul, double dip the pipette tips in the wells to remove the ‘hanging drop’.

Replace the lid and seal in an irradiated pouch. (Item number WIPSS4 Steriking sterilization Pouches)

It is possible to stack the dispensed plates before loading into the pouch.

Store all the plates at 2-8° C. for between 15-24 hours.

Transfer the plates to −20° C. on stainless steel shelves in the Cuddons freezer for at least 8 hours. Ensure the shelves/trays are pre-frozen before use. It is possible to store the frozen plates in a freezer for up to 2 months.

Prepare the lyophilizer. (Virtis Freeze Dryer, Model: 6203-6508-9×)

Freeze dry the plates on pre-frozen trays and commence the cycle. The cycle is −20° C. for a minimum of 46 hours with a vacuum of approximately 10 millitorr. The drying time is a minimum of 46 hours, e.g., between 46-72 hours.

Optionally, open each pouch in a clean room and apply plate label.

Seal plate in a foil bag with a 1 g desiccant.

Optionally, label the foil pouch with a label.

Use a heat sealer to seal the end of the foil bag.

Store plates at Room Temperature until required.

### Example 8

### Culturing Cells Using a 3D Cell Culture Matrix Comprising Alginate

This example describes a procedure for culturing cells in an alginate matrix produced by the procedure described in Example 7 in 96-well plates. However, it is expected that the procedure can be generally applicable with various types of cell culture matrices.

Inoculate at low-density (e.g., 25,000 cells/well) or high-density (e.g., 300,000 cells/well) as indicated below, or optimize for specific cell types. In general, cells inoculated at 25,000 per sponge can be cultured 5 days without medium exchange while cells inoculated at 300,000 per sponge may need daily refeeding. All amounts are given on a per well basis. See the workflow to the right for an overview.

1. Remove Alginate Culture System plate from package, if relevant. Discard desiccant if present.

2. Low-density culture (25,000 cells/well): Remove cells from culture and resuspend in culture medium at a concentration of about 8.33×10⁵ cells/ml. Inoculate 30 μl of this cell suspension into the middle of each dry sponge in the 96-well plate, e.g., with an electronic 8 channel multichannel pipette.

High-density culture (300,000 cells/well): Remove cells from culture and resuspend in culture medium at a concentration of 1×10⁷ cells/ml. Inoculate 30 μl of this cell suspension into the middle of each dry sponge in the 96-well plate, e.g., with an electronic 8 channel multichannel pipette.

3. Optional: Dynamically seed the sponges by immediately centrifuging the 96-well plates at about 100×g for about 4 minutes. Note: Certain cell types may be embedded more thoroughly within the sponge with dynamic seeding.

4. Place the plate in an incubator (e.g., about 36-38° C. in a humidified atmosphere of about 4 to 6% CO₂ in air) for about 10 minutes. If using multiple plates, do not stack plates.

5. Remove plate from incubator and place in hood. Dispense 200 μl of room temperature cell culture medium into each well, e.g., using an electronic 8 channel multichannel pipette. Note: Immediately after inoculation numerous air bubbles may be present in the scaffold/matrix. This is normal; the bubbles typically disappear after 2-3 days in inoculated wells as cells consume oxygen.

6. Incubate plate(s) in an incubator (e.g., about 36-38° C. in a humidified atmosphere of about 4 to 6% CO₂ in air). To avoid edge-effect evaporation of water from the outer wells (a problem with some incubators), place plate(s) on a moistened paper towel in a container covered with perforated aluminum-foil.

7. For high-density culture: Replenish medium daily by gently withdrawing 150 μl of medium and adding an equivalent amount of fresh medium. Cultures inoculated at lower densities may need media replacement when media turns yellow. Note: Do not allow the pipettor tips to contact the bioscaffold when withdrawing spent medium. Keep the tips on an angle against the wall of the well to avoid sucking up the sponge/matrix. If loose bioscaffolds interfere with media refeeding, add 100 μl of medium to each well, centrifuge the plates at 400×g for 7 minutes, then withdraw excess medium, repeating once if desired.

8. After several days, remove plate from incubator and exam under light microscopy (e.g., low magnification) for presence of spheroid formation.

### Example 8

### PAA pH Titration and Alginate Sponge Adherence Experiment

Methods:

1. 1% PAA in water, distributed into 3 tubes, pH to 3.20, 7.28 and 9.55.

2. Added 0.5 ml PAA per well of a 24 well polystyrene tissue culture plate and incubate 30 minutes at room temperature.

3. Either withdrew PAA with no washes or withdrew PAA followed by 2 washes (0.5 ml each).

4. After PAA removal, plates were dried under a hood with lids removed (2 hours).

5. Hydrated 96 well sponges (prepared as in Example 7, except the 96 well plates were not coated with PAA) and transferred to the center of the wells of a 24 well polystyrene tissue culture tray. Six sponges were tested per condition

6. Two ml of RPMI 1640+10% FBS was added to each well of the 24 well plates containing a sponge.

7. The number of sponges remaining attached for each condition after 105 minutes was recorded.

The results are shown in Table 10. The level of adherence with increased stringency imposed by washing, increased with increasing pH.

TABLE 10

| | | # of Matrices |
|---|---|---|
| Condition | Wash conditions | Attached |
| No PAA | No wash | 0 |
| No PAA | Two washes | 0 |
| PAA - Acidic pH 3.2 | No wash | 6 |
| PAA - Acidic pH 3.2 | Two washes | 1 |
| PAA - Acidic pH 7.28 | No wash | 6 |
| PAA - Acidic pH 7.28 | Two washes | 2 |
| PAA - Acidic pH 9.55 | No wash | 6 |
| PAA - Acidic pH 9.55 | Two washes | 4 |

All publications, patents and patent applications mentioned in this specification are herein incorporated by reference in their entirety into the specification to the same extent as if each individual publication, patent or patent application was specifically and individually indicated to be incorporated herein by reference.

1. A composition comprising a first surface coated with a charged molecule and a second surface adhering to the first surface, wherein a cell culture matrix comprises the second surface.

2. The composition of claim 1, wherein the first and the second surfaces are negatively charged and the charged molecule is a positively charged molecule.

3. The composition of claim 2, wherein a tissue culture vessel comprises the first surface.

4. The composition of claim 3, wherein the positively charged molecule is a polyamine.

5. The composition of claim 4, wherein the polyamine is a polyallylamine.

6. The composition of claim 5, wherein the cell culture matrix is a sponge.

7. The composition of claim 6, wherein the sponge is comprised of an alginate.

8. A method of producing a composition, the method comprising:
(a) coating a first surface with a positively charged molecule; and
(b) contacting the first surface with a second surface, wherein a cell culture matrix comprises the second surface.

9. The method of claim 8, wherein a tissue culture vessel comprises the first surface.

10. The method of claim 9, wherein the coating of the first surface comprises contacting the first surface with a positively charged molecule in a solvent.

11. The method of claim 10, wherein the positively charged molecule is a polyamine.

12. The method of claim 11, wherein the polyamine is a polyallylamine.

13. The method of claim 12, wherein the cell culture matrix is a sponge.

14. The method of claim 13, wherein the sponge is comprised of an alginate.

15. A method of culturing cells on a cell culture matrix, the method comprising:
(a) coating a first surface with a positively charged molecule;
(b) contacting the first surface with a second surface, wherein a cell culture matrix comprises the second surface; and
(c) contacting the cells with the cell culture matrix under conditions suitable for culturing the cells.

16. The method of claim 15, wherein a tissue culture vessel comprises the first surface.

17. The method of claim 16, wherein the coating of the first surface comprises contacting the first surface with a positively charged molecule in a solvent.

18. The method of claim 17, wherein the positively charged molecule is a polyamine.

19. The method of claim 18, wherein the polyamine is a polyallylamine.

20. The method of claim 19, wherein the cell culture matrix is a sponge.

21. The method of claim 20, wherein the sponge is comprised of an alginate.

22. A method of determining an effect of at least one compound on a cell comprising:
(a) coating a first surface with a positively charged molecule;
(b) contacting the first surface with a second surface, wherein a cell culture matrix comprises the second surface;
(c) contacting the cells with the cell culture matrix under conditions suitable for culturing the cells;
(d) contacting the cells of (c) with the at least one compound; and
(e) determining or detecting the effect or lack of effect of the at least one compound on the cell.

23. The method of claim 22, wherein a tissue culture vessel comprises the first surface.

24. The method of claim 23, wherein the coating of the first surface comprises contacting the first surface with a positively charged molecule in a solvent.

25. The method of claim 24, wherein the positively charged molecule is a polyamine.

26. The method of claim 25, wherein the polyamine is a polyallylamine.

27. The method of claim 26, wherein the cell culture matrix is a sponge.

28. The method of claim 27, wherein the sponge is comprised of an alginate.

29. A method of producing a cell culture matrix, the method comprising contacting a first surface with the cell culture matrix, wherein the cell culture matrix comprises a positively charged molecule.

30. A method of culturing cells on a cell culture matrix, the method comprising:
(a) contacting a first surface with the cell culture matrix, wherein the cell culture matrix comprises a positively charged molecule; and
(b) contacting the cells with the cell culture matrix under conditions suitable for culturing the cells.

31. A method of determining an effect of at least one compound on a cell comprising:
(a) contacting a first surface with a cell culture matrix, wherein the cell culture matrix comprises a positively charged molecule;
(b) contacting the cells with the cell culture matrix under conditions suitable for culturing the cells;
(c) contacting the cells of (b) with the at least one compound; and
(d) determining or detecting the effect or lack of effect of the at least one compound on the cell.

32. A method of adhering a cell to a surface comprising:
(a) coating a surface with a polyallylamine; and
(b) contacting the surface with a cell.
